(19) United States
(12) Patent Application Publication
(10) Pub. No.: US 2021/0128634 A1
(43) Pub. Date: May 6, 2021

(54) EXOSOMES DERIVED FROM CORTICAL BONE STEM CELLS CAN AUGMENT HEART FUNCTION AFTER CARDIAC INJURY

(71) Applicant: Temple University-Of The Commonwealth System of Higher Education, Philadelphia, PA (US)

(72) Inventors: Steven R. Houser, Fort Washington, PA (US); Hajime Kubo, North Wales, PA (US); Sadia Mohsin, Sewell, NJ (US)

(21) Appl. No.: 16/635,687

(22) PCT Filed: Aug. 1, 2018
(86) PCT No.: PCT/US18/44730

§ 371 Date: Jan. 31, 2020

Related U.S. Application Data
(60) Provisional application No. 62/539,612, filed on Aug. 1, 2017.

(51) Int. Cl. A61K 35/32; C12N 5/0775; C12N 15/113; A61P 9/10

(52) U.S. Cl. CPC A61K 35/32; C12N 5/0662; C12N 2310/141; A61P 9/10; C12N 15/113

(57) ABSTRACT

The present invention provides an isolated population of cortical bone stem cell (CBSC)-derived exosomes, and compositions comprising the exosomes and/or RNA thereof, for promoting cardiac repair when delivered to a diseased heart.

### CROSS REFERENCE TO RELATED APPLICATIONS

This application claims priority to U.S. Provisional Application No. 62/539,612, filed Aug. 1, 2017 which is hereby incorporated by reference herein in its entirety.

### BACKGROUND OF THE INVENTION

Ischemic injury of the heart, including myocardial infarction (MI), is a major health problem that leads to structural and functional remodeling (Pfeffer et al., 1990, Circulation 81:1161-1172) and often culminates in heart failure (Gomez et al., 2001, Circulation 104:688-693). Novel therapies to repair or replace damaged cardiac tissue are needed to improve the prognosis of MI patients. Stem cell therapy has the potential to repair hearts after ischemic injury.

The mechanisms of stem cell-mediated cardiac repair are unknown. Many preclinical studies in animal models have shown that differentiation (Smith et al., 2007, Circulation 115:896-908; Orlic et al., 2001, Nature 410:701-705; Rota et al., 2007, Proc Natl Acad Sci USA 104:17783-17788) of injected cells into new cardiac myocytes is one potential mechanism for this repair. Studies have shown that transplantation of autologous cardiac (Beltrami et al., 2003, Cell 114:763-776; Tang et al., 2010, Circulation 121:293-305; Rota et al., 2008, Circ Res 103:107-116; Smith et al., 2007, Circulation 115:896-908) or bone marrow-derived (Orlic et al., 2001, Nature 410:701-705; Rota et al., 2007, Proc Natl Acad Sci USA 104:17783-17788) stem cells, induced pluripotent stem cells (Mauritz et al., 2008, Circulation 118:507-517; Zhang et al., 2009, Circ Res 104:e30-41; Zwi et al., 2009, Circulation 120:1513-1523), and direct reprogramming of endogenous non-stem cells into cardiogenic phenotypes (Qian et al., 2012, Nature 485:593-598; Song et al, 2012, Nature 485:599-604) can improve cardiac function after injury. However, major limitations of this approach are the diminished proliferation, survival and differentiation capacity of the donated stem cell population and the cells' ability to integrate within the host environment.

Therefore, there is a need in the art for compositions and methods for cell-free therapeutic methods to enhance cardiac repair. The present invention addresses this unmet need.

### SUMMARY OF THE INVENTION

In one aspect, the invention provides a composition for treating a cardiovascular disease or disorder in a subject, comprising an isolated cortical bone stem cell (CBSC)-derived exosome.

In one embodiment, the exosome composition further comprises at least one RNA molecule.

In one embodiment, the RNA molecule of the exosome composition is at least one selected from the group: miR-142, miR-16, miR-21, miR-124, miR-126, miR-15, miR-29, miR-9, let-7, miR-24, miR-27, miR-30, miR-22, miR-140, miR-155, miR-130, miR-322, miR-17, miR-125, miR-29, miR-872, miR-32, miR-19, miR-191, miR-126, miR-93, miR-146, miR-196, miR-30, miR-18, miR-28, miR-23, miR-150, miR-92, miR-10, miR-106, miR-34, miR-503, miR-25, miR-96, miR-31, miR-15, miR-10, miR-144, miR-467, miR-99, miR-880, miR-199, miR-488, miR-182, miR-291, miR-186, miR-541, miR-302, miR-183, miR-411, miR-295, miR-1, miR-214, miR-138, miR-425, miR-218, miR-335, miR-101, miR-141, miR-744, miR-39, miR-142a-5p, miR-16-5p, miR-142a-3p, miR-21a-5p, miR-124-3p, miR-126a-3p, miR-15a-5p, miR-29b-3p, miR-9-5p, let-7c-5p, miR-24-3p, miR-27a-3p, miR-30e-5p, miR-22-3p, miR-30a-5p, let-7a-5p, miR-30d-5p, miR-140-5p, let-7f-5p, miR-155-5p, miR-130a-3p, let-7b-5p, miR-322-5p, miR-17-5p, miR-27b-3p, miR-125b-5p, miR-29a-3p, miR-872-5p, miR-32-5p, miR-19b-3p, miR-191-5p, miR-126a-5p, miR-93-5p, miR-146a-5p, miR-196b-5p, let-7i-5p, miR-20a-5p, miR-18a-5p, miR-28c, miR-23b-3p, miR-150-5p, miR-92a-3p, miR-10a-5p, let-7d-5p, miR-196a-5p, miR-23a-3p, miR-106b-5p, miR-34c-5p, miR-503-5p, miR-25-3p, miR-7g-5p, miR-96-5p, miR-31-5p, miR-30c-5p, miR-15b-5p, miR-10b-5p, miR-144-3p, miR-467e-5p, miR-125a-5p, miR-99a-5p, miR-880-3p, miR-19a-3p, miR-199a-5p, miR-488-3p, miR-182-5p, miR-291a-3p, miR-186-5p, miR-541-5p, miR-302d-3p, miR-183-5p, let-7e-5p, miR-140-3p, miR-411-5p, miR-295-3p, miR-1a-3p, miR-214-3p, miR-138-5p, miR-425-5p, miR-218-5p, miR-335-5p, miR-101a-3p, miR-141-3p, miR-744-5p, miR-467c-5p, miR-39-3p, SNORD61, SNORD68, SNORD72, SNORD95, SNORD96A, RNU6-6P, a variant, a derivative, and a combination thereof.

In another aspect, the invention provides a composition for treating a cardiovascular disease or disorder in a subject, comprising at least one RNA molecule.

In one embodiment, the RNA molecule is at least one selected from the group: miR-142, miR-16, miR-21, miR-124, miR-126, miR-15, miR-29, miR-9, let-7, miR-24, miR-27, miR-30, miR-22, miR-140, miR-155, miR-130, miR-322, miR-17, miR-125, miR-29, miR-872, miR-32, miR-19, miR-191, miR-126, miR-93, miR-146, miR-196, miR-30, miR-18, miR-28, miR-23, miR-150, miR-92, miR-10, miR-106, miR-34, miR-503, miR-25, miR-96, miR-31, miR-15, miR-10, miR-144, miR-467, miR-99, miR-880, miR-199, miR-488, miR-182, miR-291, miR-186, miR-541, miR-302, miR-183, miR-411, miR-295, miR-1, miR-214, miR-138, miR-425, miR-218, miR-335, miR-101, miR-141, miR-744, miR-39, miR-142a-5p, miR-16-5p, miR-142a-3p, miR-21a-5p, miR-124-3p, miR-126a-3p, miR-15a-5p, miR-29b-3p, miR-9-5p, let-7c-5p, miR-24-3p, miR-27a-3p, miR-30e-5p, miR-22-3p, miR-30a-5p, let-7a-5p, miR-30d-5p, miR-140-5p, let-7f-5p, miR-155-5p, miR-130a-3p, let-7b-5p, miR-322-5p, miR-17-5p, miR-27b-3p, miR-125b-5p, miR-29a-3p, miR-872-5p, miR-32-5p, miR-19b-3p, miR-191-5p, miR-126a-5p, miR-93-5p, miR-146a-5p, miR-196b-5p, let-7i-5p, miR-20a-5p, miR-18a-5p, miR-28c, miR-23b-3p, miR-150-5p, miR-92a-3p, miR-10a-5p, let-7d-5p, miR-196a-5p, miR-23a-3p, miR-106b-5p, miR-34c-5p, miR-503-5p, miR-25-3p, miR-7g-5p, miR-96-5p, miR-31-5p, miR-30c-5p, miR-15b-5p, miR-10b-5p, miR-144-3p, miR-467e-5p, miR-125a-5p, miR-99a-5p, miR-880-3p, miR-19a-3p, miR-199a-5p, miR-488-3p, miR-182-5p, miR-291a-3p, miR-186-5p, miR-541-5p, miR-302d-3p, miR-183-5p, let-7e-5p, miR-140-3p, miR-411-5p, miR-295-3p, miR-1a-3p, miR-214-3p, miR-138-5p, miR-425-5p, miR-218-5p, miR-335-5p, miR-101a-3p, miR-141-3p, miR-744-5p, miR-467c-5p, miR-39-3p, SNORD61, SNORD68, SNORD72, SNORD95, SNORD96A, RNU6-6P, a variant, a derivative, and a combination thereof.

In one aspect, the invention provides a method of treating at least one cardiovascular disease or disorder in a subject, comprising administering to said subject a therapeutically effective amount of a composition comprising at least one selected from the group: a CBSC-derived exosome and an RNA molecule.

In one embodiment, the RNA molecule of the method is at least one selected from the group: miR-142, miR-16, miR-21, miR-124, miR-126, miR-15, miR-29, miR-9, let-7, miR-24, miR-27, miR-30, miR-22, miR-140, miR-155, miR-130, miR-322, miR-17, miR-125, miR-29, miR-872, miR-32, miR-19, miR-191, miR-126, miR-93, miR-146, miR-196, miR-30, miR-18, miR-28, miR-23, miR-150, miR-92, miR-10, miR-106, miR-34, miR-503, miR-25, miR-96, miR-31, miR-15, miR-10, miR-144, miR-467, miR-99, miR-880, miR-199, miR-488, miR-182, miR-291, miR-186, miR-541, miR-302, miR-183, miR-411, miR-295, miR-1, miR-214, miR-138, miR-425, miR-218, miR-335, miR-101, miR-141, miR-744, miR-39, miR-142a-5p, miR-16-5p, miR-142a-3p, miR-21a-5p, miR-124-3p, miR-126a-3p, miR-15a-5p, miR-29b-3p, miR-9-5p, let-7c-5p, miR-24-3p, miR-27a-3p, miR-30e-5p, miR-22-3p, miR-30a-5p, let-7a-5p, miR-30d-5p, miR-140-5p, let-7f-5p, miR-155-5p, miR-130a-3p, let-7b-5p, miR-322-5p, miR-17-5p, miR-27b-3p, miR-125b-5p, miR-29a-3p, miR-872-5p, miR-32-5p, miR-19b-3p, miR-191-5p, miR-126a-5p, miR-93-5p, miR-146a-5p, miR-196b-5p, let-7i-5p, miR-20a-5p, miR-18a-5p, miR-28c, miR-23b-3p, miR-150-5p, miR-92a-3p, miR-10a-5p, let-7d-5p, miR-196a-5p, miR-23a-3p, miR-106b-5p, miR-34c-5p, miR-503-5p, miR-25-3p, miR-7g-5p, miR-96-5p, miR-31-5p, miR-30c-5p, miR-15b-5p, miR-10b-5p, miR-144-3p, miR-467e-5p, miR-125a-5p, miR-99a-5p, miR-880-3p, miR-19a-3p, miR-199a-5p, miR-488-3p, miR-182-5p, miR-291a-3p, miR-186-5p, miR-541-5p, miR-302d-3p, miR-183-5p, let-7e-5p, miR-140-3p, miR-411-5p, miR-295-3p, miR-1a-3p, miR-214-3p, miR-138-5p, miR-425-5p, miR-218-5p, miR-335-5p, miR-101a-3p, miR-141-3p, miR-744-5p, miR-467c-5p, miR-39-3p, SNORD61, SNORD68, SNORD72, SNORD95, SNORD96A, RNU6-6P, a variant, a derivative, and a combination thereof.

In one embodiment, said cardiovascular disease is myocardial injury.

In one embodiment, said myocardial injury is at least one selected from the group: arterial disease, atheroma, atherosclerosis, arteriosclerosis, coronary artery disease, arrhythmia, angina pectoris, congestive heart disease, ischemic cardiomyopathy, myocardial infarction, stroke, transient ischemic attack, aortic aneurysm, cardiopericarditis, infection, inflammation, valvular insufficiency, vascular clotting defects, and a combination thereof.

In one embodiment, said composition is administered to said subject by at least one selected from the group: direct injection, venous infusion, and arterial infusion.

In one embodiment, a composition of the invention further comprises a pharmaceutically acceptable excipient, carrier, or diluent.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1, comprising FIG. 1A through FIG. 1J, depicts exemplary experimental results demonstrating exosomes isolated from CBSCs. FIG. 1A depicts bone derived stem cell in culture. FIG. 1B depicts a micrograph of CBSC-derived exosome demonstrating typical morphology and size <100 nm. FIG. 1C depicts dynamic light scattering (DLS) confirming the size range of the small vesicle size. FIG. 1D through FIG. 1F is a set of images depicting TUNEL staining in neonatal rat ventricular myocytes (NRVMs). FIG. 1D depicts TUNEL staining in non-treated NRVMs. FIG. 1E depicts TUNEL staining in NRVMs exposed to apoptotic injury. FIG. 1F depicts NRVMs pretreated with exosomes derived from CBSCs and exposed to apoptotic challenge. FIG. 1G is a quantification of the results of FIG. 1D through FIG. 1F.

FIG. 2, comprising FIG. 2A through FIG. 2F, depicts exemplary experimental results demonstrating that transplantation of CBSC-derived exosomes has functional benefit after myocardial injury. FIG. 2A depicts an analysis of the ejection fraction percentage measured by echocardiography in animals treated with parent cells and animals treated with exosomes derived from CBSCs. FIG. 2B depicts an analysis of the percentage of fractional shortening measured by echocardiography in animals treated with parent cells and animals treated with exosomes derived from CBSCs. FIG. 2C depicts images of infarcts in animals treated with parent cells and animals treated with exosomes derived from CBSCs. FIG. 2D depicts an analysis of the percentage of infarct size in animals treated with parent cells and animals treated with exosomes derived from CBSCs. FIG. 2E depicts images of TUNEL stained cells two days post MI. FIG. 2F depicts exemplary results demonstrating the quantification of TUNEL stained cells two days post MI. No significant differences in cardiac function were identified between animals treated with parent cells and animals treated with exosomes derived from CBSCs.

FIG. 3 depicts images demonstrating infarct size in in animals treated with parent cells and animals treated with exosomes derived from CBSCs 6 weeks after transplantation post MI.

FIG. 4 depicts images demonstrating that mice injected with exosomes have increased vessel density.

FIG. 5, comprising FIG. 5A through FIG. 5D, depicts exemplary experimental results demonstrating that CBSCs and CBSCs derived exosomes modulate the native immune response after cardiac injury. FIG. 5A depicts an mRNA expression analysis of anti-inflammatory factors in the border zone of animals treated with CBSCs and CBSCs derived exosomes. FIG. 5B depicts an exemplary serum analysis of anti-inflammatory factors in CBSCs and CBSCs-exosome treated animals. FIG. 5C depicts a histological analysis of CD86 in CBSCs and CBSCs-exosome treated animals. FIG. 5D depicts a quantification of the serum analysis of FIG. 5B.

FIG. 6, comprising FIG. 6A through FIG. 6G, depicts exemplary experimental results demonstrating that CBSC exosomes enhance cardiac function by promoting cardiomyocyte survival and modulating the cardiac immune response in the heart post-MI. FIG. 6A depicts a diagram demonstrating the strategy for isolation of cardiac immune cells. FIG. 6B depicts an analysis of the expression of pan-hematopoietic marker CD45 in CBSCs derived exosomes and saline administered animals 7 days post MI, measured by FACS. FIG. 6C depicts the quantification of CD206 expression in CBSCs derived exosomes and saline administered animals 7 days post MI. FIG. 6D depicts the quantification of CD8 expression in CBSCs derived exosomes and saline administered animals 7 days post MI. FIG. 6E depicts an analysis of the expression of pan-hematopoietic marker CD45 in CBSCs derived exosomes and saline administered animals 14 days post MI, measured by FACS. FIG. 6F depicts the quantification of CD206 expression in CBSCs derived exosomes and saline administered animals 14 days post MI. FIG. 6G depicts the quantification of CD8 expression in CBSCs derived exosomes and saline administered animals 14 days post MI.

FIG. 7, comprising FIG. 7A through FIG. 7D, depicts exemplary experimental results demonstrating that the cardiac immune response is modulated by CBSC exosomes. FIG. 7A depicts an analysis of the expression of CD3+ cells after CBSCs transplantation in the heart 14 days post MI. FIG. 7B depicts an analysis of the expression of foxp3+ cells after CBSCs transplantation in the heart 14 days post MI. FIG. 7C depicts an analysis of the expression of foxp3+ cells after CBSCs transplantation in the heart 14 days post MI. FIG. 7C depicts exemplary flow cytometry analyses of CD8+ and CD4+ cells. FIG. 7D depicts a quantification of CD8+ and CD4+ cells after CBSCs or CBSCs-exo transplantation in the heart 14 days post MI.

FIG. 8, comprising FIG. 8A through FIG. 8B, depicts exemplary experimental results demonstrating the capacity of CBSC exosomes for immune modulation in vitro. FIG. 8A depicts an analysis of pro-inflammatory factors in isolated macrophages from the bone marrow (BMDMΦs) which were co-cultured in a trans-well system with CBSCs. FIG. 8B depicts an analysis of the level of phagocytosis in BMDMΦs treated with CBSCs medium compared to LPS treatment.

FIG. 9, comprising FIG. 9A through FIG. 9B, depicts exemplary experimental results demonstrating expression of different miRNA (miR) in exosomes. FIG. 9A depicts expression of miR in exosomes derived from CBSCs compared to the corresponding CBSC. FIG. 9B depicts the comparison of miR in exosomes derived from Endothelial progenitor cells (EPC) and cortical bone derived stem cells (CBSC) (dark grey is high and light grey is low expression).

FIG. 10, comprising FIG. 10A through FIG. 10C, depicts exemplary experimental results demonstrating analyses of treatment in miniswine post MI and 1 month post MI. FIG. 10A depicts NOGA Maps at baseline for placebo treated miniswine. FIG. 10B depicts NOGA Maps at baseline for CBSC treated miniswine. FIG. 10C depicts experimental results showing animals treated with CBSCs had significantly reduced scar size 1 month post MI as compared to the placebo treated group.

### DETAILED DESCRIPTION

The present invention provides cortical bone-derived stem cell (CBSC)-derived exosomes and compositions derived thereof. In one embodiment, the cells from which the exosomes are derived are pluripotent. In another embodiment, the cells from which the exosomes are derived are capable of differentiating into cardiac myocytes. The present invention also includes methods of using CBSC-derived exosomes in the treatment of heart disease.

The invention is based partly on the discovery that CBSC-derived exosomes injected into the border zone of an induced myocardial infarction (MI) resulted in significant improvements in cardiac structure, function and survival. CBSC-derived exosomes enhanced angiogenesis in the MI border zone after MI by providing factors that trigger endogenous blood vessel formation. In addition, CBSC-derived exosomes injected into the MI border zone differentiated into new, functionally mature heart muscle cells that enhanced cardiac function in the regions of cell injection.

In one embodiment, the invention provides a novel population of exosomes derived from stem cells derived from cortical bone (e.g., CBSCs) whereby the cells may be c-kit+ and Sca1+ but may not express hematopoietic lineage markers. In one embodiment, expression of c-kit by CBSCs is decreased following subsequent culturing. However, CBSCs may continue to express all of the other signature markers from early to late passage including but is not limited to CD29, Sca-1, CD105, CD106, CD73, CD44, CD271 and CD90. CBSCs may remain negative for hematopoietic lineage markers including but is not limited to CD45, and CD11b.

In another embodiment, the CBSC-derived exosomes of the invention can function when injected into the ischemic heart and have the potential to promote production of cardiac myocytes with mature function and to provide factors that promote endogenous repair.

### Definitions

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

The term “bioreactor” is to be given its usual meaning in the art, i.e. an apparatus used to carry out a bioprocess. The bioreactors described herein are suitable for use in CBSC culture. Simple bioreactors for cell culture are single compartment flasks, such as the commonly-used T-175 flask. Bioreactors can have multiple compartments, as is known in the art. These multi-compartment bioreactors typically contain at least two compartments separated by one or more membranes or barriers that separate the compartment containing the cells from one or more compartments containing gas and/or culture medium. Multi-compartment bioreactors are well-known in the art. An example of a multi-compartment bioreactor is the Integra CeLLine bioreactor, which contains a medium compartment and a cell compartment separated by means of a 10 kDa semi-permeable membrane; this membrane allows a continuous diffusion of nutrients into the cell compartment with a concurrent removal of any inhibitory waste product. The individual accessibility of the compartments allows one to supply cells with fresh medium without mechanically interfering with the culture. A silicone membrane forms the cell compartment base and provides an optimal oxygen supply and control of carbon dioxide levels by providing a short diffusion pathway to the cell compartment. Any suitable multi-compartment bioreactor may be used.

“Cardiomyocyte” refers to cells that comprise the heart and are also known as cardiac muscle cells. A “myoblast” is a mononucleated, undifferentiated muscle precursor cell.

As used herein, the phrase “cardiovascular condition, disease or disorder” is intended to include all disorders characterized by insufficient, undesired or abnormal cardiac function, e.g., ischemic heart disease, hypertensive heart disease and pulmonary hypertensive heart disease, valvular disease, congenital heart disease and any condition which leads to congestive heart failure in a subject, particularly a human subject. Insufficient or abnormal cardiac function can be the result of disease, injury and/or aging. By way of background, a response to myocardial injury follows a well-defined path in which some cells die while others enter a state of hibernation where they are not yet dead but are dysfunctional. This is followed by infiltration of inflammatory cells, deposition of collagen as part of scarring, all of which happen in parallel with in-growth of new blood vessels and a degree of continued cell death. As used herein, the term “ischemia” refers to any localized tissue ischemia due to reduction of the inflow of blood. The term “myocardial ischemia” refers to circulatory disturbances caused by coronary atherosclerosis and/or inadequate oxygen supply to the myocardium. For example, an acute myocardial infarction represents an irreversible ischemic insult to myocardial tissue. This insult results from an occlusive (e.g., thrombotic or embolic) event in the coronary circulation and produces an environment in which the myocardial metabolic demands exceed the supply of oxygen to the myocardial tissue.

The terms “cells” and “population of cells” are used interchangeably and refer to a plurality of cells, i.e., more than one cell. The population may be a pure population comprising one cell type. Alternatively, the population may comprise more than one cell type. In the present invention, there is no limit on the number of cell types that a cell population may comprise.

As used herein, “a cell that differentiates into a mesodermal (or ectodermal or endodermal) lineage” defines a cell that becomes committed to a specific mesodermal, ectodermal or endodermal lineage, respectively. Examples of cells that differentiate into a mesodermal lineage or give rise to specific mesodermal cells include, but are not limited to, cells that are adipogenic, chondrogenic, cardiogenic, dermatogenic, hematopoetic, hemangiogenic, myogenic, nephrogenic, urogenitogenic, osteogenic, pericardiogenic, or stromal. Examples of cells that differentiate into ectodermal lineage include, but are not limited to epidermal cells, neurogenic cells, and neurogliagenic cells. Examples of cells that differentiate into endodermal lineage include, but are not limited to pleurigenic cells, and hepatogenic cells, cell that give rise to the lining of the intestine, and cells that give rise to pancreogenic and splanchogenic cells.

As used herein “conditioned media” defines a medium in which a specific cell or population of cells have been cultured in, and then removed. While the cells were cultured in said medium, they secrete cellular factors that include, but are not limited to hormones, cytokines, extracellular matrix (ECM), proteins, vesicles, antibodies, and granules. The medium plus the cellular factors is the conditioned medium.

“Differentiated” is used herein to refer to a cell that has achieved a terminal state of maturation such that the cell has developed fully and demonstrates biological specialization and/or adaptation to a specific environment and/or function. Typically, a differentiated cell is characterized by expression of genes that encode differentiation associated proteins in that cell. When a cell is said to be “differentiating,” as that term is used herein, the cell is in the process of being differentiated.

“Differentiation medium” is used herein to refer to a cell growth medium comprising an additive or a lack of an additive such that a stem cell, adipose derived adult stromal cell or other such progenitor cell, that is not fully differentiated when incubated in the medium, develops into a cell with some or all of the characteristics of a differentiated cell.

The term “derived from” is used herein to mean to originate from a specified source.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

An “effective amount” or “therapeutically effective amount” of a compound is that amount of compound which is sufficient to provide a beneficial effect to the subject to which the compound is administered. An “effective amount” of a delivery vehicle is that amount sufficient to effectively bind or deliver a compound.

As used herein “growth factors” is intended the following non-limiting factors including, but not limited to, growth hormone, erythropoietin, thrombopoietin, interleukin 3, interleukin 6, interleukin 7, macrophage colony stimulating factor, c-kit ligand/stem cell factor, osteoprotegerin ligand, insulin, insulin like growth factors, epidermal growth factor (EGF), fibroblast growth factor (FGF), nerve growth factor, ciliary neurotrophic factor, platelet derived growth factor (PDGF), transforming growth factor (TGF-beta), hepatocyte growth factor (HGF), and bone morphogenetic protein at concentrations of between picogram/ml to milligram/ml levels.

As used herein, the term “growth medium” is meant to refer to a culture medium that promotes growth of cells. A growth medium will generally contain animal serum. In some instances, the growth medium may not contain animal serum.

An “isolated cell” refers to a cell which has been separated from other components and/or cells which may accompany the isolated cell in a tissue or mammal.

As used herein, the “lineage” of a cell defines the heredity of the cell, i.e.; which cells it came from and what cells it can give rise to. The lineage of a cell places the cell within a hereditary scheme of development and differentiation.

The term “microparticle” is known in the art and encompasses a number of different species of microparticle, including a membrane particle, membrane vesicle, microvesicle, exosome-like vesicle, exosome, ectosome-like vesicle, ectosome or exovesicle. The different types of microparticle are distinguished based on diameter, subcellular origin, their density in sucrose, shape, sedimentation rate, lipid composition, protein markers and mode of secretion (i.e. following a signal (inducible) or spontaneously (constitutive)).

A “multi-lineage stem cell” or “multipotent stem cell” refers to a stem cell that reproduces itself and at least two further differentiated progeny cells from distinct developmental lineages. The lineages can be from the same germ layer (i.e. mesoderm, ectoderm or endoderm), or from different germ layers. An example of two progeny cells with distinct developmental lineages from differentiation of a multi-lineage stem cell is a myogenic cell and an adipogenic cell (both are of mesodermal origin, yet give rise to different tissues). Another example is a neurogenic cell (of ectodermal origin) and adipogenic cell (of mesodermal origin).

As used herein, the term “myocardial injury” or “injury to myocardium” refers to any structural or functional disorder, disease, or condition that affects the heart and/or blood vessels. Examples of myocardial injury can include, but are not limited to, arterial disease, atheroma, atherosclerosis, arteriosclerosis, coronary artery disease, arrhythmia, angina pectoris, congestive heart disease, ischemic cardiomyopathy, myocardial infarction, stroke, transient ischemic attack, aortic aneurysm, cardiopericarditis, infection, inflammation, valvular insufficiency, vascular clotting defects, and combinations thereof.

As used herein, a “pluripotent cell” defines a less differentiated cell that can give rise to at least two distinct (genotypically and/or phenotypically) further differentiated progeny cells.

The terms “precursor cell,” “progenitor cell,” and “stem cell” are used interchangeably in the art and herein and refer either to a pluripotent, or lineage-uncommitted, progenitor cell, which is potentially capable of an unlimited number of mitotic divisions to either renew itself or to produce progeny cells which will differentiate into the desired cell type. Unlike pluripotent stem cells, lineage-committed progenitor cells are generally considered to be incapable of giving rise to numerous cell types that phenotypically differ from each other. Instead, progenitor cells give rise to one or possibly two lineage-committed cell types.

“Proliferation” is used herein to refer to the reproduction or multiplication of similar forms, especially of cells. That is, proliferation encompasses production of a greater number of cells, and can be measured by, among other things, simply counting the numbers of cells, measuring incorporation of 3H-thymidine into the cell, and the like.

“Progression of or through the cell cycle” is used herein to refer to the process by which a cell prepares for and/or enters mitosis and/or meiosis. Progression through the cell cycle includes progression through the G1 phase, the S phase, the G2 phase, and the M-phase.

A cell may be characterized as “positive” for a particular biomarker. A cell positive for a biomarker is one wherein a cell of the invention expresses a specific biomarker protein, or a nucleic acid encoding said protein.

A cell may be characterized as “negative” for a particular biomarker. A cell negative for a biomarker is one wherein a cell of the invention does not express a detectable specific biomarker protein, or a nucleic acid encoding said protein.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, a cell exists in a “purified form” when it has been isolated away from all other cells that exist in its native environment, but also when the proportion of that cell in a mixture of cells is greater than would be found in its native environment. Stated another way, a cell is considered to be in “purified form” when the population of cells in question represents an enriched population of the cell of interest, even if other cells and cell types are also present in the enriched population. A cell can be considered in purified form when it comprises at least about 10% of a mixed population of cells, at least about 20% of a mixed population of cells, at least about 25% of a mixed population of cells, at least about 30% of a mixed population of cells, at least about 40% of a mixed population of cells, at least about 50% of a mixed population of cells, at least about 60% of a mixed population of cells, at least about 70% of a mixed population of cells, at least about 75% of a mixed population of cells, at least about 80% of a mixed population of cells, at least about 90% of a mixed population of cells, at least about 95% of a mixed population of cells, or about 100% of a mixed population of cells, with the proviso that the cell comprises a greater percentage of the total cell population in the “purified” population that it did in the population prior to the purification. In this respect, the terms “purified” and “enriched” can be considered synonymous.

“Self-renewal” refers to the ability to produce replicate daughter stem cells having differentiation potential that is identical to those from which they arose. A similar term used in this context is “proliferation.”

As used herein, “stem cell” defines an undifferentiated cell that can produce itself and/or a further differentiated progeny cell.

As used herein, “tissue engineering” refers to the process of generating tissues ex vivo for use in tissue replacement or reconstruction. Tissue engineering is an example of “regenerative medicine,” which encompasses approaches to the repair or replacement of tissues and organs by incorporation of cells, gene or other biological building blocks, along with bioengineered materials and technologies.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention relates to exosomes for therapeutic use in treatment of cardiac diseases, disorders, and injuries. The invention also relates to the therapeutic use of one or more RNA molecules for treatment of cardiac injury. In one embodiment, the RNA molecules are packaged in an exosome. In one embodiment, the exosome is a CBSC-derived exosome.

Without wishing to be bound by any particular theory, it is believed that exosomes play a role in intercellular communication by acting as vehicles between a donor and recipient cell through direct and indirect mechanisms. Direct mechanisms include the uptake of the exosome and its donor cell-derived components (such as proteins, lipids or nucleic acids) by the recipient cell, the components having a biological activity in the recipient cell. Indirect mechanisms include exosome-recipient cell surface interaction, and causing modulation of intracellular signaling of the recipient cell. Hence, exosomes may mediate the acquisition of one or more donor cell-derived properties by the recipient cell. It has been observed that, despite the efficacy of stem cell therapies in animal models, the stem cells do not appear to engraft into the host. Accordingly, the mechanism by which stem cell therapies are effective is not clear. Without wishing to be bound by a particular theory, it is believed that the exosome secreted by the stem cells play a role in the therapeutic utility of these cells and are therefore therapeutically useful themselves.

Generally, the exosomes of the invention are isolated. The term “isolated” indicates that the exosome or exosome population to which it refers is not within its natural environment. The exosome or exosome population has been substantially separated from surrounding cells and/or tissue. In some embodiments, the exosome or exosome population is substantially separated from surrounding cells and/or tissue if the sample contains at least about 75%, in some embodiments at least about 85%, in some embodiments at least about 90%, and in some embodiments at least about 95% exosomes. In other words, the sample is substantially separated from the surrounding tissue if the sample contains less than about 25%, in some embodiments less than about 15%, and in some embodiments less than about 5% of materials other than the exosomes. Such percentage values refer to percentage by weight. The term encompasses exosomes which have been removed from the organism from which they originated, and exist separately. The term also encompasses exosomes which have been removed from the organism from which they originated, and subsequently re-inserted into an organism. The organism which contains the re-inserted cells may be the same organism from which the cells were removed, or it may be a different organism.

Typically, the cortical bone stem cell (CBSC) population from which the exosomes are produced, is substantially pure. The term “substantially pure” as used herein, refers to a population of CBSCs that is at least about 75%, in some embodiments at least about 85%, in some embodiments at least about 90%, and in some embodiments at least about 95% pure, with respect to other cells that make up a total cell population. For example, with respect to CBSC populations, this term means that there are at least about 75%, in some embodiments at least about 85%, in some embodiments at least about 90%, and in some embodiments at least about 95% pure, CBSCs compared to other cells that make up a total cell population. In other words, the term “substantially pure” refers to a population of CBSCs of the present invention that contain fewer than about 25%, in some embodiments fewer than about 15%, and in some embodiments fewer than about 5%, of lineage committed cells in the original unamplified and isolated population prior to subsequent culturing and amplification.

A CBSC exosome comprises at least one lipid bilayer which typically encloses a milieu comprising lipids, proteins and nucleic acids. The nucleic acids may be deoxyribonucleic acid (DNA) and/or ribonucleic acid (RNA). RNA may be messenger RNA (mRNA), micro RNA (miRNA, miR) or any miRNA precursors, such as pri-miRNA, pre-miRNA, and/or small nuclear RNA (snRNA).

A CBSC-derived exosome retains at least one biological function of the CBSC from which it is derived. Biological functions that may be retained include the ability to promote regeneration of cardiac tissues. In one embodiment, the at least one biological function is that of a CBSC that has been cultured in a multi-compartment bioreactor, for at least 10 weeks and optionally no more than 20 weeks. Alternatively the at least one biological function may be that of a CBSC-conditioned medium from a CBSC population that has been cultured in a multi-compartment bioreactor, for at least 10 weeks and optionally no more than 20 weeks. In another embodiment, the at least one biological function is that of a CBSC that has been cultured in a cell culture flask under standard conditions.

In one embodiment, the RNA of the composition is a miR and/or snoRNA. In another embodiment, the RNA is contained within CBSC-derived exosomes and can be isolated therefrom. In one embodiment, exosomes for providing the miR and/or snoRNAs for therapeutic use in treatment of cardiac injury are artificial exosomes.

In one embodiment, CBSC-derived exosomes, or miR and/or snoRNAs derived therefrom are useful for wound repair, in vivo and ex vivo tissue regeneration, tissue transplantation, and other methods that require miR or snoRNAs that are provided by the exosomes of the invention.

In one embodiment, the RNA is at least one selected from the group: miR-142a-5p, miR-16-5p, miR-142a-3p, miR-21a-5p, miR-124-3p, miR-126a-3p, miR-15a-5p, miR-29b-3p, miR-9-5p, let-7c-5p, miR-24-3p, miR-27a-3p, miR-30e-5p, miR-22-3p, miR-30a-5p, let-7a-5p, miR-30d-5p, miR-140-5p, let-7f-5p, miR-155-5p, miR-130a-3p, let-7b-5p, miR-322-5p, miR-17-5p, miR-27b-3p, miR-125b-5p, miR-29a-3p, miR-872-5p, miR-32-5p, miR-19b-3p, miR-191-5p, miR-126a-5p, miR-93-5p, miR-146a-5p, miR-196b-5p, let-7i-5p, miR-20a-5p, miR-18a-5p, miR-28c, miR-23b-3p, miR-150-5p, miR-92a-3p, miR-10a-5p, let-7d-5p, miR-196a-5p, miR-23a-3p, miR-106b-5p, miR-34c-5p, miR-503-5p, miR-25-3p, miR-7g-5p, miR-96-5p, miR-31-5p, miR-30c-5p, miR-15b-5p, miR-10b-5p, miR-144-3p, miR-467e-5p, miR-125a-5p, miR-99a-5p, miR-880-3p, miR-19a-3p, miR-199a-5p, miR-488-3p, miR-182-5p, miR-291a-3p, miR-186-5p, miR-541-5p, miR-302d-3p, miR-183-5p, let-7e-5p, miR-140-3p, miR-411-5p, miR-295-3p, miR-1a-3p, miR-214-3p, miR-138-5p, miR-425-5p, miR-218-5p, miR-335-5p, miR-101a-3p, miR-141-3p, miR-744-5p, miR-467c-5p, miR-39-3p, SNORD61, SNORD68, SNORD72, SNORD95, SNORD96A, RNU6-6P, a variant, a derivative, and a combination thereof.

In a particular embodiment, the RNA is at least one selected from the group: miR-142a-5p, miR-16-5p, miR-142a-3p, miR-124-3p, miR-126a-3p, miR-15a-5p, miR-29b-3p, miR-9-5p, let-7c-5p, let-7a-5p, miR-140-5p, let-7f-5p, miR-155-5p, miR-130a-3p, let-7b-5p, miR-322-5p, miR-17-5p, miR-125b-5p, miR-29a-3p, miR-872-5p, miR-32-5p, miR-19b-3p, miR-126a-5p, miR-196b-5p, let-7i-5p, miR-18a-5p, miR-28c, miR-23b-3p, miR-10a-5p, let-7d-5p, miR-196a-5p, miR-23a-3p, miR-34c-5p, miR-503-5p, miR-7g-5p, miR-96-5p, miR-31-5p, miR-30c-5p, miR-10b-5p, miR-144-3p, miR-467e-5p, miR-125a-5p, miR-99a-5p, miR-880-3p, miR-199a-5p, miR-488-3p, miR-182-5p, miR-291a-3p, miR-186-5p, miR-541-5p, miR-302d-3p, miR-183-5p, let-7e-5p, miR-140-3p, miR-411-5p, miR-295-3p, miR-1a-3p, miR-214-3p, miR-138-5p, miR-425-5p, miR-218-5p, miR-335-5p, miR-101a-3p, miR-141-3p, miR-467c-5p, miR-39-3p, SNORD61, SNORD68, SNORD72, RNU6-6P, a variant, a derivative, and a combination thereof.

In one embodiment, the RNA comprises one or more members of one or more miRNA gene families. In one embodiment, the RNA is at least one selected from the group: miR-142, miR-16, miR-21, miR-124, miR-126, miR-15, miR-29, miR-9, let-7, miR-24, miR-27, miR-30, miR-22, miR-140, miR-155, miR-130, miR-322, miR-17, miR-125, miR-29, miR-872, miR-32, miR-19, miR-191, miR-126, miR-93, miR-146, miR-196, miR-30, miR-18, miR-28, miR-23, miR-150, miR-92, miR-10, miR-106, miR-34, miR-503, miR-25, miR-96, miR-31, miR-15, miR-10, miR-144, miR-467, miR-99, miR-880, miR-199, miR-488, miR-182, miR-291, miR-186, miR-541, miR-302, miR-183, miR-411, miR-295, miR-1, miR-214, miR-138, miR-425, miR-218, miR-335, miR-101, miR-141, miR-744, miR-39, SNORD61, SNORD68, SNORD72, SNORD95, SNORD96A, RNU6-6P, a variant, a derivative, and a combination thereof.

In one embodiment, the RNA is at least one selected from the group: miR-142, miR-16, miR-21, miR-124, miR-126, miR-15, miR-29, miR-9, let-7, miR-24, miR-27, miR-30, miR-22, miR-140, miR-155, miR-130, miR-322, miR-17, miR-125, miR-29, miR-872, miR-32, miR-19, miR-191, miR-126, miR-93, miR-146, miR-196, miR-30, miR-18, miR-28, miR-23, miR-150, miR-92, miR-10, miR-106, miR-34, miR-503, miR-25, miR-96, miR-31, miR-15, miR-10, miR-144, miR-467, miR-99, miR-880, miR-199, miR-488, miR-182, miR-291, miR-186, miR-541, miR-302, miR-183, miR-411, miR-295, miR-1, miR-214, miR-138, miR-425, miR-218, miR-335, miR-101, miR-141, miR-744, miR-39, SNORD61, SNORD68, SNORD72, SNORD95, SNORD96A, RNU6-6P, miR-142a-5p, miR-16-5p, miR-142a-3p, miR-21a-5p, miR-124-3p, miR-126a-3p, miR-15a-5p, miR-29b-3p, miR-9-5p, let-7c-5p, miR-24-3p, miR-27a-3p, miR-30e-5p, miR-22-3p, miR-30a-5p, let-7a-5p, miR-30d-5p, miR-140-5p, let-7f-5p, miR-155-5p, miR-130a-3p, let-7b-5p, miR-322-5p, miR-17-5p, miR-27b-3p, miR-125b-5p, miR-29a-3p, miR-872-5p, miR-32-5p, miR-19b-3p, miR-191-5p, miR-126a-5p, miR-93-5p, miR-146a-5p, miR-196b-5p, let-7i-5p, miR-20a-5p, miR-18a-5p, miR-28c, miR-23b-3p, miR-150-5p, miR-92a-3p, miR-10a-5p, let-7d-5p, miR-196a-5p, miR-23a-3p, miR-106b-5p, miR-34c-5p, miR-503-5p, miR-25-3p, miR-7g-5p, miR-96-5p, miR-31-5p, miR-30c-5p, miR-15b-5p, miR-10b-5p, miR-144-3p, miR-467e-5p, miR-125a-5p, miR-99a-5p, miR-880-3p, miR-19a-3p, miR-199a-5p, miR-488-3p, miR-182-5p, miR-291a-3p, miR-186-5p, miR-541-5p, miR-302d-3p, miR-183-5p, let-7e-5p, miR-140-3p, miR-411-5p, miR-295-3p, miR-1a-3p, miR-214-3p, miR-138-5p, miR-425-5p, miR-218-5p, miR-335-5p, miR-101a-3p, miR-141-3p, miR-744-5p, miR-467c-5p, miR-39-3p, a variant, a derivative, and a combination thereof.

In one embodiment, the invention provides a novel population of purified exosomes from CBSCs.

### Compositions

The present invention provides an isolated CBSC-derived exosome, compositions comprising a CBSC-derived exosome, and compositions comprising at least one RNA molecule. The isolated CBSC-derived exosomes of the present composition can be obtained from any mammalian source, including, but not limited to, humans, primates, canines, felines, bovines, ovines, porcines, equines, and rodents. In addition, the CBSC-derived exosomes can be autologous or heterologous, with respect to the subject to whom they are administered. The CBSC-derived exosomes may be, but are not necessarily, derived from CBSC obtained from the subject to whom the CBSC-derived exosomes are then administered. In some embodiments, the CBSC-derived exosomes are obtained from one or more individuals other than the patient (i.e., heterologous CBSC-derived exosomes). In certain embodiments, the CBSC-derived exosomes originate from a pool of CBSC-derived exosomes derived from CBSC obtained from two or more donors.

In one embodiment, the concentration of CBSC-derived exosomes in a composition described herein may be greater than 10¹ CBSC-derived exosomes/μL, greater than 10² CBSC-derived exosomes/μL, greater than 10³ CBSC-derived exosomes/μL, greater than 10⁴ CBSC-derived exosomes/μL, greater than 10⁵ CBSC-derived exosomes/μL, greater than 10⁶ CBSC-derived exosomes/μL, greater than 10⁷ CBSC-derived exosomes/μL, greater than 10⁸ CBSC-derived exosomes/μL, greater than 10⁹ CBSC-derived exosomes/μL, greater than 10¹⁰ CBSC-derived exosomes/μL, greater than 10¹¹ CBSC-derived exosomes/μL, greater than 10¹² CBSC-derived exosomes/μL, greater than 10¹³ CBSC-derived exosomes/μL, or greater than 10¹⁴ CBSC-derived exosomes/μL. In one embodiment, the concentration of CBSC-derived exosomes in a composition described herein may be less than 10¹ CBSC-derived exosomes/μL, less than 10² CBSC-derived exosomes/μL, less than 10³ CBSC-derived exosomes/μL, less than 10⁴ CBSC-derived exosomes/μL, less than 10⁵ CBSC-derived exosomes/μL, less than 10⁶ CBSC-derived exosomes/μL, less than 10⁷ CBSC-derived exosomes/μL, less than 10⁸ CBSC-derived exosomes/μL, less than 10⁹ CBSC-derived exosomes/μL, less than 10¹⁰ CBSC-derived exosomes/μL, less than 10¹¹ CBSC-derived exosomes/μL, less than 10¹² CBSC-derived exosomes/μL, less than 10¹³ CBSC-derived exosomes/μL, or less than 10¹⁴ CBSC-derived exosomes/μL.

The CBSC-derived exosomes of the compositions described herein may be subjected to various conditions prior to use in treating a subject. They can be concentrated by any suitable method, including, but not limited to, centrifugation and filtration. In addition to concentration, they can be washed one or more times with saline or another suitable solution to purify the CBSC-derived exosomes. Likewise, they can be maintained as a packed concentrate, having little or essentially no liquid medium surrounding them, or suspended in a suitable aqueous solution or buffer that may contain stabilizers or other substances that are compatible with CBSC-derived exosomes. They can also be filtered or prepared from a filtered product and can be pathogen treated to inactivate a broad spectrum of viruses and bacteria, a process intended to reduce the risk of transfusion transmitted infections that is useful in various applications.

### Genetic Modification

In another embodiment, the CBSC-derived exosomes of the invention can be derived from CBSCs that have been genetically modified, e.g., to express exogenous (e.g., introduced) genes (“transgenes”) or to repress the expression of endogenous genes. In accordance with this method, the CBSC-derived exosomes of the invention can be derived from CBSCs that have been exposed to a gene transfer vector comprising a nucleic acid comprising a transgene, such that the nucleic acid is introduced into the cell under conditions appropriate for the transgene to be expressed within the cell. The transgene generally may be an expression cassette, including a polynucleotide operably linked to a suitable promoter. The polynucleotide can encode a protein, or it can encode biologically active RNA (e.g., antisense RNA or a ribozyme). Where it is desired to employ gene transfer technology to deliver a given transgene, the transgene sequence will generally be known.

Such genetic modification may have therapeutic benefit. Alternatively, the genetic modification may provide a means to track or identify the cells so-modified, for instance, after implantation of a composition of the invention into an individual. Tracking an exosome-targeted cell may include tracking the function of a transplanted genetically-modified cell-derived exosome. Genetic modification may also include at least a second gene. A second gene may encode, for instance, a selectable antibiotic-resistance gene or another selectable marker.

Viral and non-viral vector means of genetic modification of CBSCs, in vitro, in vivo and ex vivo are included. Introduction of compositions (e.g., nucleic acid and protein) into the cells can be carried out by methods known in the art, such as osmotic shock (e.g., calcium phosphate), electroporation, microinjection, cell fusion, etc. Introduction of nucleic acid and polypeptide in vitro, ex vivo and in vivo can also be accomplished using other techniques. For example, this can be accomplished by use of a polymeric substance, such as polyesters, polyamine acids, hydrogel, polyvinyl pyrrolidone, ethylene-vinylacetate, methylcellulose, carboxymethylcellulose, protamine sulfate, or lactide/glycolide copolymers, polylactide/glycolide copolymers, or ethylenevinylacetate copolymers. A nucleic acid can be entrapped in microcapsules prepared by coacervation techniques or by interfacial polymerization, for example, using hydroxymethylcellulose or gelatin-microcapsules, or poly (methylmethacrolate) microcapsules, respectively, or in a colloid system. Colloidal dispersion systems include macromolecule complexes, nano-capsules, microspheres, beads, and lipid-based systems, including oil-in-water emulsions, micelles, mixed micelles, and liposomes.

Liposomes for introducing various compositions into cells are known in the art and include, for example, phosphatidylcholine, phosphatidylserine, lipofectin and DOTAP (e.g., U.S. Pat. Nos. 4,844,904, 5,000,959, 4,863,740, and 4,975,282; and GIBCO-BRL, Gaithersburg, Md.). Piperazine-based amphiphilic cationic lipids useful for gene therapy also are known (see, e.g., U.S. Pat. No. 5,861,397). Cationic lipid systems also are known (see, e.g., U.S. Pat. No. 5,459,127). Polymeric substances, microcapsules and colloidal dispersion systems such as liposomes may be collectively referred to herein as “vesicles.”

Exosomes may retain at least some of the functions of the CBSCs that produce them. Therefore, it is possible to design exosomes by manipulating the stem cell (which can be any stem cell type and is not limited to cortical bone stem cells) to possess one or more desired functions, typically expression of protein or miRNA. The manipulation will typically involve genetic engineering, to introduce one or more exogenous coding, non-coding or regulatory nucleic acid sequences into the CBSC. For example, if an exosome containing VEGF and/or bFGF is desired, then the exosome-producing CBSC can be transformed or transfected to express (high levels of) VEGF and/or bFGF, which would then be incorporated into the exosomes produced by that CBSC. The invention therefore includes ad hoc exosomes, from any stem cell type, that contain a function that is not naturally present in the cell from which they are produced, i.e. the exosomes may contain one or more exogenous protein or nucleic acid sequences, which are not naturally-occurring and which are engineered.

In one embodiment, isolated or purified exosomes from the conditioned medium of CBSCs that have been cultured are loaded with one or more exogenous nucleic acids, lipids, proteins, drugs or prodrugs which are intended to perform a desired function in a target cell. This does not require manipulation of the CBSC and the exogenous material can optionally be directly added to the exosomes. For example, exogenous nucleic acids can be introduced into the exosomes by electroporation. The exosomes can then be used as vehicles or carriers for the exogenous material. In one embodiment, exosomes that have been isolated from the cells that produced them are loaded with exogenous siRNA, typically by electroporation, to produce exosomes that can be deployed to silence one or more pathological genes. In this way, exosomes can be used as vehicles to deliver one or more agents, typically therapeutic or diagnostic agents, to a target cell, for example to enhance or complement their endogenous inhibition of heart disease progression. An example of this is a CBSC exosome comprising exogenous siRNA capable of silencing one or more pathological genes.

### Methods of Obtaining Exosomes of the Invention

Cortical bone tissue can be used as a source of CBSC-derived exosomes of the invention. In one embodiment, the CBSC-derived exosomes of the invention are capable of promoting cardiac repair. In one embodiment, the CBSC-derived exosomes of the invention are capable of promoting myogenesis. In another embodiment, the CBSC-derived exosomes of the invention are capable of promoting angiogenesis. Accordingly, the CBSC-derived exosomes of the present invention can be used to treat cardiac tissue damaged due to injury or disease. It is understood by those of skill in the art that the term treating, as used herein, includes directly or indirectly repairing, replacing, augmenting, improving, rescuing, repopulating, or regenerating.

Exosomes may be isolated from CBSC conditioned media. The “conditioned medium” (CM) may be a growth medium for CBSCs, which has been used to culture a mass culture of CBSCs, removed and sterilized by any suitable means, for example by filtration, prior to use, if required.

Exosomes that are able to treat cardiac disease or disorder have been isolated from CBSCs that have been cultured for a sufficient period. Accordingly, one way to produce exosomes is to culture the cells in a multi-compartment bioreactor for a sufficient period before the exosomes are harvested, for example at least 1 day, at least 2 days, at least 3 days, at least 4 days, at least 5 days, at least 6 days, at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, and optionally no longer than 20 weeks. In one embodiment, the CBSCs are cultured for any period determined to be suitable for producing exosomes.

The exosomes may be separated from other media components based on molecular weight, size, shape, hydrodynamic radius, composition, charge, substrate-ligand interaction, absorbance or scattering of electromagnetic waves, or biological activity. In one embodiment, the conditioned media is filtered using a filter of appropriate size to separate the desired exosome, for example a 100 K MWCO filter. Optionally, the conditioned medium is concentrated prior to the isolation of the exosomes by subjecting the concentrated conditioned medium to size exclusion chromatography. The UV absorbant fractions can then be selected for isolation of the exosomes of interest.

Different types of exosomes can be isolated from the media by using different isolation techniques and parameters. For example, exosomes have a vesicle density of 1.13-1.19 g/mL and can be isolated by differential centrifugation and sucrose gradient ultracentrifugation at 100,000-200,000 g.

A typical production method comprises: culturing CBSCs to produce conditioned media; removing cell debris by centrifugation at 1500 rpm; isolating exosomes by ultrafiltration or isolating exosomes by ultracentrifugation at 120,000 g; and quantifying exosome content using a BCA protein assay.

The cells for producing exosomes may be obtained from a subject that is relatively young, for example, at an age that is at most one tenths, one fifths, one third, or half of the subject's expected life span. For example, the exosomes may be obtained from a human that is at most, less than or about one, two, three, four, five, six, seven, eight, nine, ten, 11, 12 months, or 1, 2, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 years old, or any age or range derivable therein. In a particular aspect, the exosomes may be obtained from a human that is less than one year old or less than 18 years old. In a particular aspect, the exosomes may be obtained from a human that is between 18 and 50 years old. The human may be the same patient that is to be treated.

Furthermore, in some aspects, the isolated exosomes or nanovesicles (e.g., the artificially engineered exosomes from in vitro reconstitution) may contain endogenous exosomes or may be loaded with externally added therapeutic agents, such as nucleic acids or protein molecules. The nucleic acids may be DNA or RNA, such as siRNA, miRNA, or mRNA. In certain aspects, the isolated exosomes may comprise RNAs such as one or more of miR-142, miR-16, miR-21, miR-124, miR-126, miR-15, miR-29, miR-9, let-7, miR-24, miR-27, miR-30, miR-22, miR-140, miR-155, miR-130, miR-322, miR-17, miR-125, miR-29, miR-872, miR-32, miR-19, miR-191, miR-126, miR-93, miR-146, miR-196, miR-30, miR-18, miR-28, miR-23, miR-150, miR-92, miR-10, miR-106, miR-34, miR-503, miR-25, miR-96, miR-31, miR-15, miR-10, miR-144, miR-467, miR-99, miR-880, miR-199, miR-488, miR-182, miR-291, miR-186, miR-541, miR-302, miR-183, miR-411, miR-295, miR-1, miR-214, miR-138, miR-425, miR-218, miR-335, miR-101, miR-141, miR-744, miR-39, miR-142a-5p, miR-16-5p, miR-142a-3p, miR-21a-5p, miR-124-3p, miR-126a-3p, miR-15a-5p, miR-29b-3p, miR-9-5p, let-7c-5p, miR-24-3p, miR-27a-3p, miR-30e-5p, miR-22-3p, miR-30a-5p, let-7a-5p, miR-30d-5p, miR-140-5p, let-7f-5p, miR-155-5p, miR-130a-3p, let-7b-5p, miR-322-5p, miR-17-5p, miR-27b-3p, miR-125b-5p, miR-29a-3p, miR-872-5p, miR-32-5p, miR-19b-3p, miR-191-5p, miR-126a-5p, miR-93-5p, miR-146a-5p, miR-196b-5p, let-7i-5p, miR-20a-5p, miR-18a-5p, miR-28c, miR-23b-3p, miR-150-5p, miR-92a-3p, miR-10a-5p, let-7d-5p, miR-196a-5p, miR-23a-3p, miR-106b-5p, miR-34c-5p, miR-503-5p, miR-25-3p, miR-7g-5p, miR-96-5p, miR-31-5p, miR-30c-5p, miR-15b-5p, miR-10b-5p, miR-144-3p, miR-467e-5p, miR-125a-5p, miR-99a-5p, miR-880-3p, miR-19a-3p, miR-199a-5p, miR-488-3p, miR-182-5p, miR-291a-3p, miR-186-5p, miR-541-5p, miR-302d-3p, miR-183-5p, let-7e-5p, miR-140-3p, miR-411-5p, miR-295-3p, miR-1a-3p, miR-214-3p, miR-138-5p, miR-425-5p, miR-218-5p, miR-335-5p, miR-101a-3p, miR-141-3p, miR-744-5p, miR-467c-5p, miR-39-3p, SNORD61, SNORD68, SNORD72, SNORD95, SNORD96A, RNU6-6P, a variant, a derivative, and a combination thereof.

### Conditionally Immortalized Stem Cells as Producer Cells For Exosomes

In one aspect of the invention, conditionally immortalized stem cells are used to produce exosomes. These conditionally immortalized stem cells are typically cortical bone derived stem cells, but may be a stem cell of any type, for example a hematopoietic stem cell or a mesenchymal stem cell. A method of producing stem cell exosomes is therefore provided, comprising the steps of culturing conditionally-immortalized stem cells and harvesting the exosomes that are produced by the cells, as described herein. Conditional immortalization of stem cells is known in the art. For the avoidance of doubt, this method is not limited to the use of CBSCs.

### Methods of Inducing Exosome Secretion

It is possible to increase the production of exosomes by stem cells. This advantage, which is not limited to cortical bone derived stem cells and can be used for the production of exosomes from any stem cell, allows for an improved yield of exosomes to be obtained from a stem cell culture.

A first technique to increase the production of exosomes by the stem cells may be to treat the stem cells with one or more of TGF-β, IFN-γ or TNF-α, typically at between 1 and 25 ng/ml e.g. 10 ng/ml, for between 12 to 96 hours prior to the removal of conditioned media.

A second technique to increase the production of exosomes by the stem cells is to culture the cells under hypoxic conditions. Culturing cells under hypoxic conditions is well-known to the skilled person, and involves culturing the cells in an atmosphere that has less than atmospheric level of O₂, i.e. less than 21% O₂. This is typically achieved by placing the cells in an incubator that allows oxygen levels to be changed. Hypoxic culture typically involves culturing in an atmosphere containing less than 10% O₂, more typically 5% or less O₂, for example 4% or less, 3% or less, 2% or less, or 1% or less O₂.

The amount of exosomes produced by stem cells can be increased greatly simply by culturing stem cells in a multi-compartment bioreactor. This property is not limited to cortical bone derived stem cells and applies generally to the culture of all stem cells. Accordingly, one aspect of the invention provides a method of producing exosomes from CBSCs that have been cultured in a multi-compartment bioreactor. The cells from which the exosomes are harvested have typically been cultured for at least one week, typically at least 8, 9, 10, 11, 12, 13 or 14 days, for example 15 days, 16 days, 17 days, 18 days, 19 days, 20 days, 21 days or more, for example at least three weeks, four weeks, five weeks, six weeks or more. To produce therapeutic exosomes, the cells from which the exosomes are harvested may have been cultured for more than ten weeks.

### Therapeutic Methods

The invention is based in part on the discovery that CBSC-derived exosomes are effective in preventing apoptosis and promoting myocardial repair when injected into the ischemic heart.

In one embodiment, the CBSC-derived exosomes of the invention are capable of promoting cardiac repair, myogenesis, angiogenesis or a combination thereof. Accordingly, the exosomes of the present invention can be used to treat cardiac tissue damaged due to injury or disease. It is understood by those of skill in the art that the term treating, as used herein, includes repairing, replacing, augmenting, improving, rescuing, repopulating, or regenerating.

Cardiovascular diseases and/or disorders include, but are not limited to, diseases and/or disorders of the pericardium, heart valves (i.e., incompetent valves, stenosed valves, rheumatic heart disease, mitral valve prolapse, aortic regurgitation), myocardium (coronary artery disease, myocardial infarction, heart failure, ischemic heart disease, angina) blood vessels (i.e., arteriosclerosis, aneurysm) or veins (i.e., varicose veins, hemorrhoids). In specific embodiments, the cardiovascular disease includes, but is not limited to, coronary artery diseases (i.e., arteriosclerosis, atherosclerosis, and other diseases of the arteries, arterioles and capillaries or related complaint), acute myocardial infarct, organizing myocardial infarct, ischemic heart disease, arrhythmia, left ventricular dilatation, emboli, heart failure, congestive heart failure, subendocardial fibrosis, left or right ventricular hypertrophy, and myocarditis. Yet further, one skilled in the art recognizes that cardiovascular diseases and/or disorders can result from congenital defects, genetic defects, environmental influences (i.e., dietary influences, lifestyle, stress, etc.), and other defects or influences.

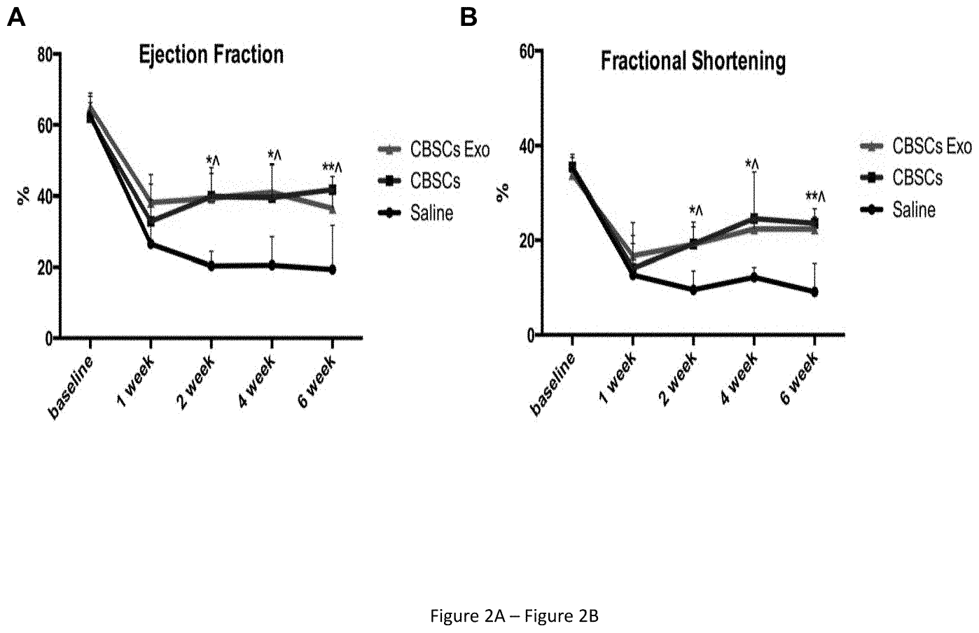

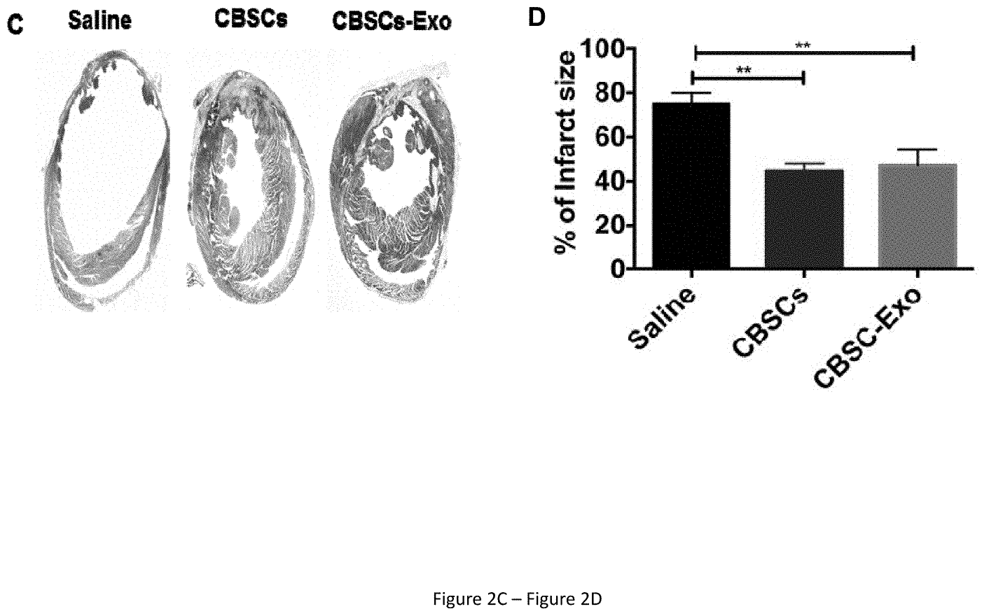

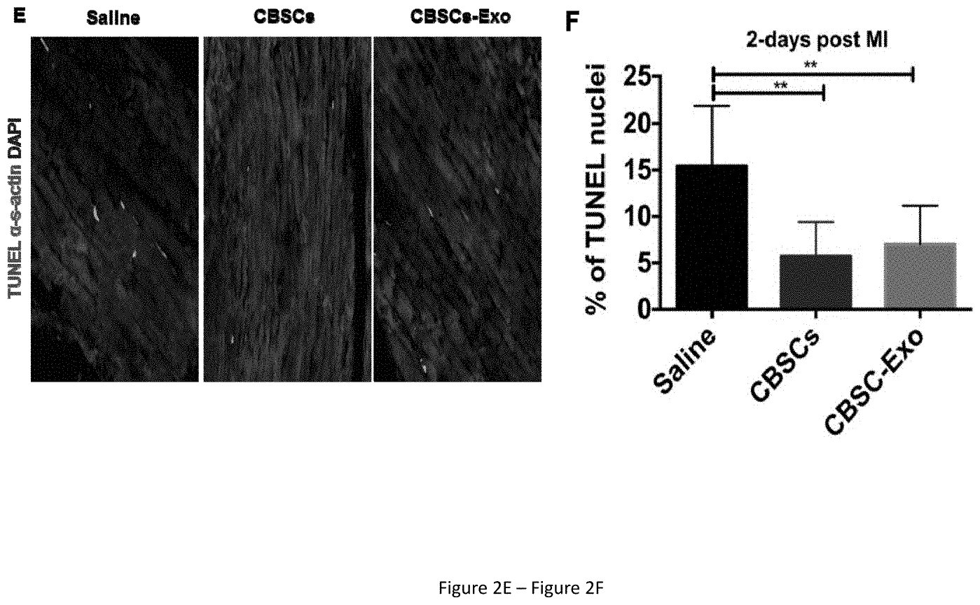

In one embodiment, the CBSC-derived exosomes of the invention can be used to treat cardiovascular diseases and disorders. CBSC-derived exosomes of the invention have several properties that can contribute to reducing and/or minimizing damage and myocyte apoptosis and promoting myocardial or cardiovascular repair and regeneration following damage. The CBSC-derived exosomes of the invention may have increased levels of expression of particular miRs, for example, as outlined in FIG. 2B.

Accordingly, in one aspect of the present invention, CBSC-derived exosomes are derived from a donor's CBSCs and are used to elicit a therapeutic benefit to damaged or degenerated myocardium or other cardiovascular tissue. Patients may be evaluated to assess myocardial damage or disease by one or more of the following procedures performed by a physician or other clinical provider: patient's health history, physical examination, and objective data including but not limited to EKG, serum cardiac enzyme profile, and echocardiography.

CBSC-derived exosomes may be administered to a patient in any setting in which myocardial function is compromised. Examples of such settings include, but are not limited to, acute myocardial infarction (heart attack), congestive heart failure (either as therapy or as a bridge to transplant), and supplementation of coronary artery bypass graft surgery, among other things. The exosomes may be collected in advance and stored in a cryopreserved fashion or they may be collected at or around the time of defined need. As disclosed herein, the exosomes may be administered to the patient, or applied directly to the damaged tissue, or in proximity of the damaged tissue, with or without further processing or following additional procedures to further purify, modify, stimulate, or otherwise change the exosomes.

The exosomes may also be applied with additives to enhance, control, or otherwise direct the intended therapeutic effect. For example, in one embodiment, the exosomes may be further purified by use of antibody-mediated positive and/or negative selection to enrich the population to increase efficacy, reduce morbidity, or to facilitate ease of the procedure. Similarly, exosomes may be applied with a biocompatible matrix which facilitates in vivo tissue engineering by supporting and/or directing the fate of the implanted exosomes.

In one embodiment, the method of the invention involves intramyocardial transplantation of CBSC-derived exosomes of the invention. Such therapeutic methods can repair and regenerate damaged myocardium and restore cardiac function after, for example, acute myocardial infarction and/or other ischemic or reperfusion related injuries. Methods generally comprise contacting a composition comprising CBSC-derived exosomes of the invention with cardiac tissue or cells.

In accordance with one method, a composition comprising CBSC-derived exosomes of the invention is introduced into the cardiac tissue or a desired site in the subject. In brief, this method can be performed as follows. CBSC-derived exosomes of the invention are isolated from cortical bone tissue. Once isolated, the CBSC-derived exosomes of the invention can be purified. The isolated CBSC-derived exosomes of the invention can then be formulated as a composition comprising the CBSC-derived exosomes of the invention along with, for example, a pharmaceutically acceptable excipient, carrier or diluent. The composition so formed can then be introduced into the heart tissue of a subject. The subject will usually have been diagnosed as having, or being at risk for, a heart condition, disease, or disorder. Introduction of the composition can be according to methods generally known to the art. For example, the CBSC-derived exosome composition can be administered to a subject's heart by way of direct injection delivery or catheter delivery. Introduction of CBSC-derived exosomes can be a single occurrence or can occur sequentially over a period selected by a physician. The time course and number of occurrences of CBSC-derived exosome implantation into a subject's heart can be dictated by monitoring generation and/or regeneration of cardiac tissue, where such methods of assessment of treatment course is within the skill of the art of an attending physician.

Cardiac tissue into which CBSC-derived exosomes of the invention can be introduced includes, but is not limited to, the myocardium of the heart (including cardiac muscle fibers, connective tissue (endomysium), nerve fibers, capillaries, and lymphatics); the endocardium of the heart (including endothelium, connective tissue, and fat cells); the epicardium of the heart (including fibroelastic connective tissue, blood vessels, lymphatics, nerve fibers, fat tissue, and a mesothelial membrane consisting of squamous epithelial cells); and any additional connective tissue (including the pericardium), blood vessels, lymphatics, fat cells, progenitor cells (e.g., side-population progenitor cells), and nervous tissue found in the heart. Cardiac muscle fibers are composed of chains of contiguous heart-muscle cells, or “cardiomyocytes”, joined end to end at intercalated disks. These disks possess two kinds of cell junctions: expanded desmosomes extending along their transverse portions, and gap junctions, the largest of which lie along their longitudinal portions. Each of the above tissues can be selected as a target site for introduction of CBSC derived exosomes, either individually or in combination with other tissues.

A determination of the need for treatment will typically be assessed by a history and physical exam consistent with the myocardial defect, disorder, or injury at issue. Subjects with an identified need of therapy include those with diagnosed damaged or degenerated heart tissue (i.e., heart tissue which exhibits a pathological condition). Causes of heart tissue damage and/or degeneration include, but are not limited to, chronic heart damage, chronic heart failure, damage resulting from injury or trauma, damage resulting from a cardiotoxin, damage from radiation or oxidative free radicals, damage resulting from decreased blood flow, and myocardial infarction (such as a heart attack). In one example, a subject in need of treatment according to the methods described herein will be diagnosed with degenerated heart tissue resulting from a myocardial infarction or heart failure. The subject may be an animal, including, but not limited to, a mammal, a reptile, and an avian, a horse, a cow, a dog, a cat, a sheep, a pig, a chicken, and a human.

It should be recognized that methods of this invention can easily be practiced in conjunction with existing myocardial therapies to effectively treat or prevent disease. The methods, compositions, and devices of the invention can include concurrent or sequential treatment with non-biologic and/or biologic drugs, surgeries, or other therapies.

The subject receiving cardiac implantation of CBSC-derived exosomes according to the methods described herein will usually have been diagnosed as having, or being at risk for, a heart condition, disease, or disorder. The methods of the invention can be useful to alleviate the symptoms of a variety of disorders, such as disorders associated with aberrant cell/tissue damage, ischemic disorders, and reperfusion related disorders. For example, the methods are useful in alleviating a symptom of myocardial infarction, chronic coronary ischemia, arteriosclerosis, congestive heart failure, dilated cardiomyopathy, restenosis, coronary artery disease, heart failure, arrhythmia, angina, atherosclerosis, hypertension, or myocardial hypertrophy. The condition, disease, or disorder can be diagnosed and/or monitored, typically by a physician using standard methodologies. Alleviation of one or more symptoms of the condition, disease, or disorder indicates that the composition confers a clinical benefit, such as a reduction in one or more of the following symptoms: shortness of breath, fluid retention, headaches, dizzy spells, chest pain, left shoulder or arm pain, and ventricular dysfunction.

Cardiac cell/tissue damage is characterized by a loss of one or more cellular functions characteristic of the cardiac cell type which can lead to eventual cell death. For example, cell damage to a cardiomyocyte results in the loss of contractile function of the cell resulting in a loss of ventricular function of the heart tissue. An ischemic or reperfusion related injury results in tissue necrosis and scar formation.

Injured myocardial tissue is defined for example by necrosis, scarring, or yellow softening of the myocardial tissue. Injured myocardial tissue leads to one or more of several mechanical complications of the heart, such as ventricular dysfunction, decreased forward cardiac output, as well as inflammation of the lining around the heart (i.e., pericarditis). Accordingly, regenerating injured myocardial tissue according to the methods described herein can result in histological and functional restoration of the tissue.

The methods of the invention can promote generation and/or regeneration of heart tissue, and/or promote endogenous myocardial regeneration of heart tissue in a subject. Promoting generation of heart tissue generally includes activating, enhancing, facilitating, increasing, inducing, initiating, or stimulating the growth and/or proliferation of heart tissue, as well as activating, enhancing, facilitating, increasing, inducing, initiating, or stimulating the differentiation, growth, and/or proliferation of heart tissue cells. Thus, the term includes initiation of heart tissue generation, as well as facilitation or enhancement of heart tissue generation already in progress. Differentiation is generally understood as the cellular process by which cells become structurally and functionally specialized during development. Proliferation and growth, as used herein, generally refer to an increase in mass, volume, and/or thickness of heart tissue, as well as an increase in diameter, mass, or number of heart tissue cells. The term generation is understood to include the generation of new heart tissue and the regeneration of heart tissue where heart tissue previously existed.

Generation of new heart tissue and regeneration of heart tissue, resultant from the therapeutic methods described herein, can be measured or detected by procedures known to the art. Such procedures include, but are not limited to, Western blotting for heart-specific proteins, electron microscopy in conjunction with morphometry, simple assays to measure rate of cell proliferation (including trypan blue staining, the CellTiter-Blue cell viability assay from Promega (Madison, Wis.), the MTT cell proliferation assay from ATCC, differential staining with fluorescein diacetate and ethidium bromide/propidium iodide, estimation of ATP levels, flow-cytometry assays, etc.), and any of the methods, molecular procedures, and assays disclosed herein.

In one embodiment, direct administration of exosomes to the site of intended benefit is exemplary. This may be achieved by direct injection into the external surface of the heart (epicardial), direct injection into the myocardium through the internal surface (endocardial) through insertion of a suitable cannula, by arterial or venous infusion (including retrograde flow mechanisms) or by other means disclosed herein or known in the art. Routes of administration known to one of ordinary skill in the art, include but are not limited to, intravenous, intracoronary, endomyocardial, epimyocardial, intraventicular, retrosinus or intravascular.

As disclosed elsewhere herein, CBSC-derived exosomes may be applied by several routes including systemic administration by venous or arterial infusion (including retrograde flow infusion) or by direct injection into the heart. Systemic administration, particularly by peripheral venous access, has the advantage of being minimally invasive relying on the natural perfusion of the heart and the ability of the CBSC-derived exosomes to target the site of damage. Exosomes may be injected in a single bolus, through a slow infusion, or through a staggered series of applications separated by several hours or, provided exosomes are appropriately stored, several days or weeks. Exosomes may also be applied by use of catheterization such that the first pass of exosomes through the heart is enhanced by using balloons to manage myocardial blood flow. As with peripheral venous access, exosomes may be injected through the catheters in a single bolus or in multiple smaller aliquots. Exosomes may also be applied directly to the myocardium by epicardial injection. This could be employed under direct visualization in the context of an open-heart procedure (such as a Coronary Artery Bypass Graft Surgery) or placement of a ventricular assist device. Catheters equipped with needles may be employed to deliver exosomes directly into the myocardium in an endocardial fashion which would allow a less invasive means of direct application.

In one embodiment, the route of delivery includes intravenous delivery through a standard peripheral intravenous catheter, a central venous catheter, or a pulmonary artery catheter. In other embodiments, the exosomes may be delivered through an intracoronary route to be accessed via currently accepted methods. The flow of exosomes may be controlled by serial inflation/deflation of distal and proximal balloons located within the patient's vasculature, thereby creating temporary no-flow zones which promote cellular engraftment or cellular therapeutic action. In another embodiment, exosomes may be delivered through an endocardial (inner surface of heart chamber) method which may require the use of a compatible catheter as well as the ability to image or detect the intended target tissue. Alternatively, exosomes may be delivered through an epicardial (outer surface of the heart) method. This delivery may be achieved through direct visualization at the time of an open-heart procedure or through a thoracoscopic approach requiring specialized exosome delivery instruments. Furthermore, exosomes could be delivered through the following routes, alone, or in combination with one or more of the approaches identified above: subcutaneous, intramuscular, sublingual, retrograde coronary perfusion, coronary bypass machinery, extracorporeal membrane oxygenation (ECMO) equipment and via a pericardial window.

In one embodiment, exosomes are administered to the patient as an intra-vessel bolus or timed infusion. In another embodiment, exosomes may be resuspended in an artificial or natural medium or tissue scaffold prior to being administered to the patient.

In one embodiment, the effects of exosome delivery therapy would be demonstrated by, but not limited to, one of the following clinical measures: increased heart ejection fraction, decreased rate of heart failure, decreased infarct size, decreased associated morbidity (pulmonary edema, renal failure, arrhythmias) improved exercise tolerance or other quality of life measures, and decreased mortality. The effects of exosome therapy can be evident over the course of days to weeks or months after the procedure. However, beneficial effects may be observed as early as several hours after the procedure, and may persist for at least several years.

Therapeutic methods may involve administering a composition containing about, at least about, or at most about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 445, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000 nanograms (ng), micrograms (μg), milligrams (mg), or grams of exosomes, or any range derivable therein. The above numerical values may also be the dosage that is administered to the patient based on the patient's weight, expressed as ng/kg, mg/kg, or g/kg, and any range derivable from those values.

Alternatively, the composition may have a concentration of exosomes that are 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000 ng/ml, μg/ml, mg/ml, or g/ml, or any range derivable therein.

The composition may be administered to (or taken by) the patient 1, 2, 3, 4, 5, 6, 7, 8, 9, 10, 11, 12, 13, 14, 15, 16, 17, 18, 19, 20 or more times, or any range derivable therein, and they may be administered every 1, 2, 3, 4, 5, 6, 7, 8, 9, 10, 11, 12, 13, 14, 15, 16, 17, 18, 19, 20, 21, 22, 23, 24 hours, or 1, 2, 3, 4, 5, 6, 7 days, or 1, 2, 3, 4, 5 weeks, or 1, 2, 3, 4, 5, 6, 7, 8, 9, 10, 11, 12 months, or any range derivable therein. It is specifically contemplated that the composition may be administered once daily, twice daily, three times daily, four times daily, five times daily, or six times daily (or any range derivable therein) and/or as needed to the patient. Alternatively, the composition may be administered every 2, 4, 6, 8, 12 or 24 hours (or any range derivable therein) to or by the patient. In some embodiments, the patient is administered the composition for a certain period of time or with a certain number of doses after experiencing symptoms of a disease or disorder.

In certain embodiments, the isolated exosomes may include one type or at least two, three, four, five, six, seven, eight, nine, ten or more different types of exosomes. The type of exosomes may be characterized by their compositions, for example, the types of nucleic acids and/or proteins of interest or effects.

### Pharmaceutical Compositions

The CBSC-derived exosome of the invention, and the RNA of the invention, are useful in therapy and can therefore be formulated as a pharmaceutical composition, alone or in combination. A pharmaceutically acceptable composition typically includes at least one pharmaceutically acceptable carrier, diluent, vehicle and/or excipient in addition to the exosomes and/or RNA of the invention. An example of a suitable carrier is Ringer's Lactate solution. A thorough discussion of such components is provided in Gennaro (2000) Remington: The Science and Practice of Pharmacy. 20th edition.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The composition, if desired, can also contain minor amounts of pH buffering agents. The carrier may comprise storage media such as Hypothermosol®, commercially available from BioLife Solutions Inc., USA. Examples of suitable pharmaceutical carriers are described in “Remington's Pharmaceutical Sciences” by E W Martin. Such compositions will contain a prophylactically or therapeutically effective amount of a prophylactic or therapeutic exosome preferably in purified form, together with a suitable amount of carrier so as to provide the form for proper administration to the subject. The formulation should suit the mode of administration. In a preferred embodiment, the pharmaceutical compositions are sterile and in suitable form for administration to a subject, preferably an animal subject, more preferably a mammalian subject, and most preferably a human subject.

The pharmaceutical composition of the invention may be in a variety of forms. These include, for example, semi-solid, and liquid dosage forms, such as lyophilized preparations, liquid solutions or suspensions, injectable and infusible solutions. The pharmaceutical composition may be injectable. A particular advantage of the exosomes of the invention is their improved robustness compared to the stem cells from which they are obtained; the exosomes can therefore be subjected to formulation, such as lyophilisation, that would not be suitable for stem cells. This is also an advantage of the RNA compositions of the invention.

It is exemplary that the methods, medicaments and compositions and exosomes of the invention are used for treating cardiac disease and/or injury, and/or for the treatment, modulation, prophylaxis, and/or amelioration of one or more symptoms associated with these diseases and disorders.

Pharmaceutical compositions will generally be in aqueous form. Compositions may include a preservative and/or an antioxidant.

To control tonicity, the pharmaceutical composition can comprise a physiological salt, such as a sodium salt. Sodium chloride (NaCl) is exemplary, which may be present at between 1 and 20 mg/ml. Other salts that may be present include potassium chloride, potassium dihydrogen phosphate, disodium phosphate dehydrate, magnesium chloride and calcium chloride.

Compositions may include one or more buffers. Typical buffers include: a phosphate buffer; a Tris buffer; a borate buffer; a succinate buffer; a histidine buffer; or a citrate buffer. Buffers will typically be included at a concentration in the 5-20 mM range. The pH of a composition will generally be between 5 and 8, and more typically between 6 and 8 e.g. between 6.5 and 7.5, or between 7.0 and 7.8.

The composition may be sterile. The composition may be gluten free. The composition may be non-pyrogenic.

The pharmaceutical composition can be administered by any appropriate route, which will be apparent to the skilled person depending on the disease or condition to be treated. Typical routes of administration include intravenous, intra-arterial, intramuscular, subcutaneous, intracranial, intranasal or intraperitoneal. For treatment of a disorder of the heart, one option is to administer the exosomes or RNA to the site of damage or disease.

The exosomes or RNA will be administered at a therapeutically or prophylactically-effective dose, which will be apparent to the skilled person. Due to the low or non-existent immunogenicity of the exosomes, it is possible to administer repeat doses without inducing a deleterious immune response.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1

### Isolation and Characterization of CBSC Exosomes

Secretion of paracrine factors that enhance cardioprotection of the endogenous myocardium, neovascularization, and recruitment of endogenous stem cells that promote repair is one possible mechanism of stem cell-mediated cardiac repair (Tang et al., 2010, Circulation 121:293-305; Rota et al., 2008, Circ Res 103:107-116; Zeng et al., 2007, Circulation 115:1866-1875; Li et al., 2012, J Am Coll Cardiol 59:942-953). Therefore, exosomes, which appear to be a major part of the paracrine effect, may provide an alternative to using cells as a therapeutic.

Exosomes are small 30-100 nm extracellular membranous vesicles that have attracted enormous interest because of their ability to modulate molecular processes in target cells (De Jong et al., 2014, Frontiers in Immunology, 5:608). Exosomes may be enriched with a variety of miRs, other noncoding RNAs and proteins that appear to be specific to the parent cells and their environmental conditions (Ung et al., 2014, Cancer Sci., 105(11):1384-92). They are also known to mediate the interaction between cells and their microenvironment (Wang et al., 2015, Oncotarget, 6(41):43992-4004). Exosomal miRs are key mediators in intercellular cross talk, particularly in cardiac conditions including myocardial infarction (MI) and heart failure (HF) (Ibrahim et al., 2015, Annu Rev Physiol., 78:67-83).

The materials and methods employed in these experiments are now described.

### Isolation of CBSC Exosomes

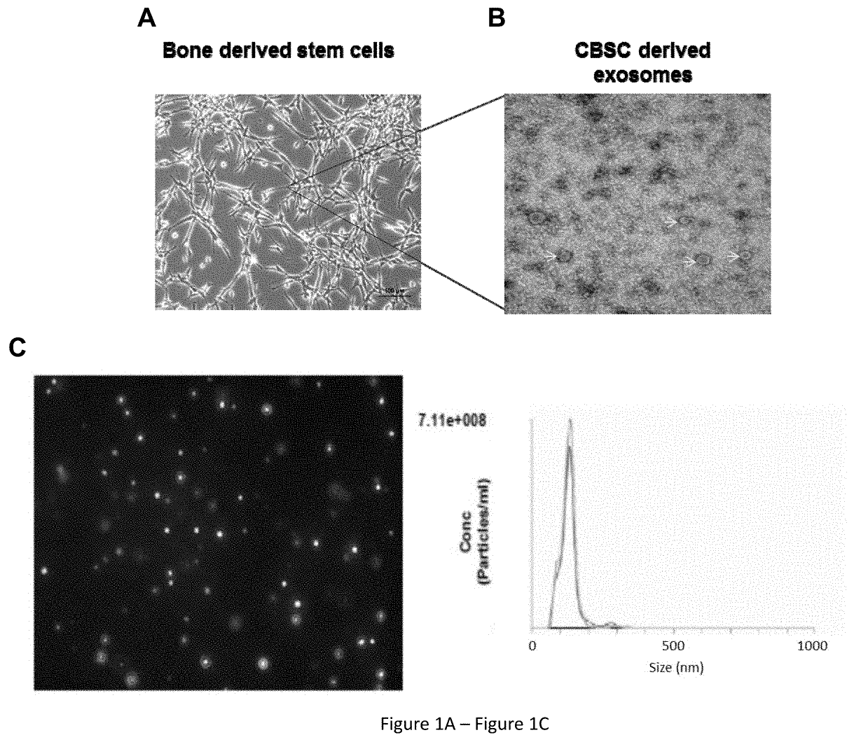

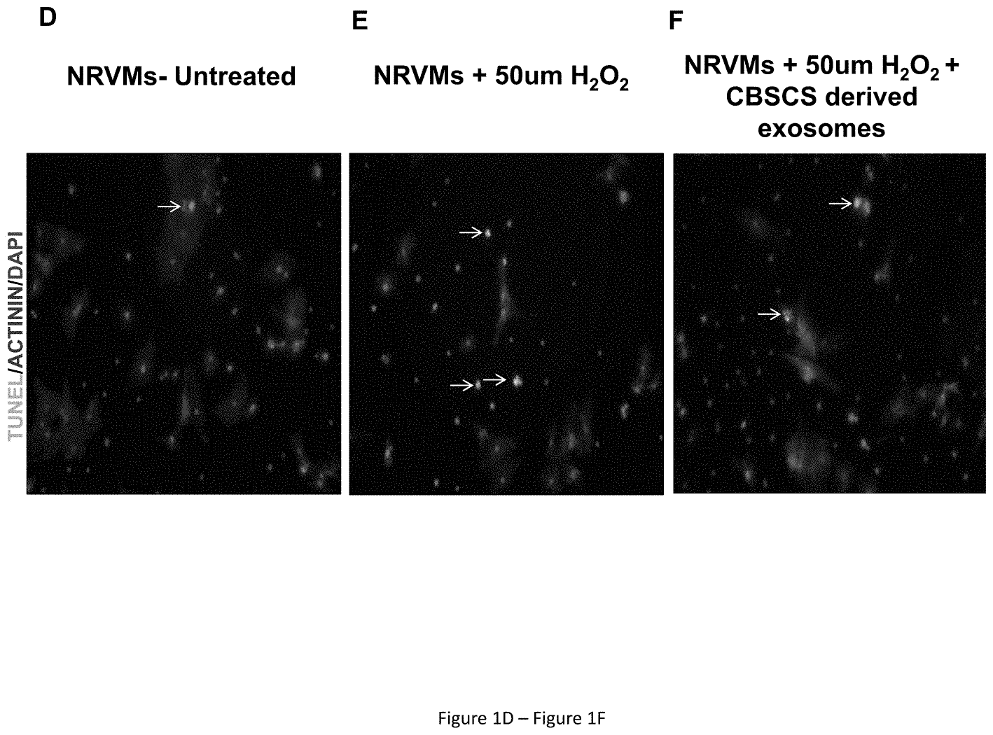

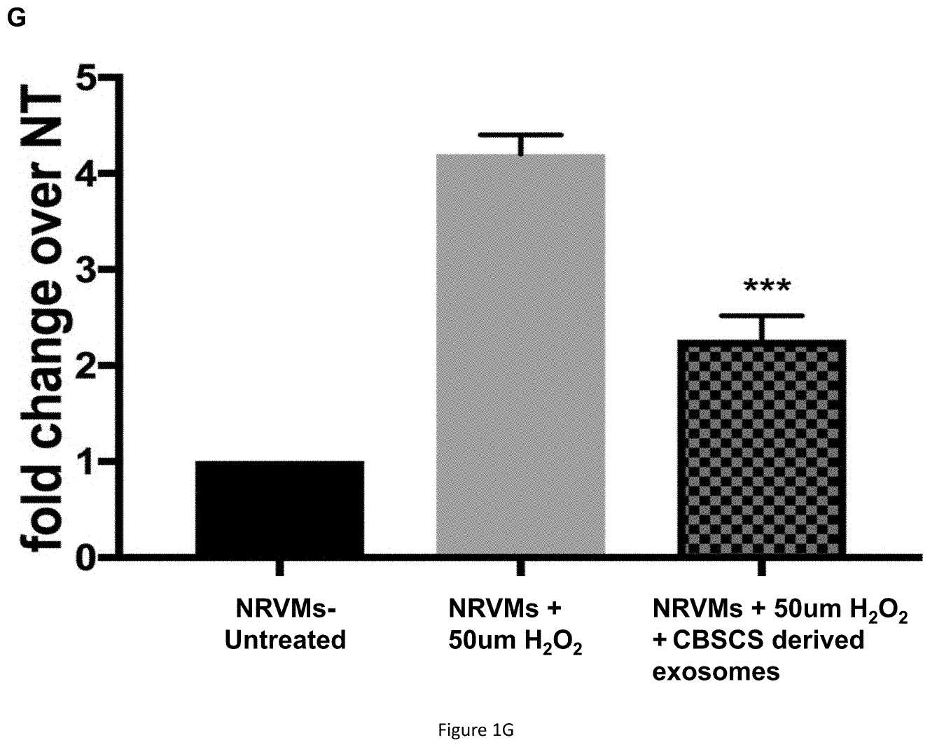

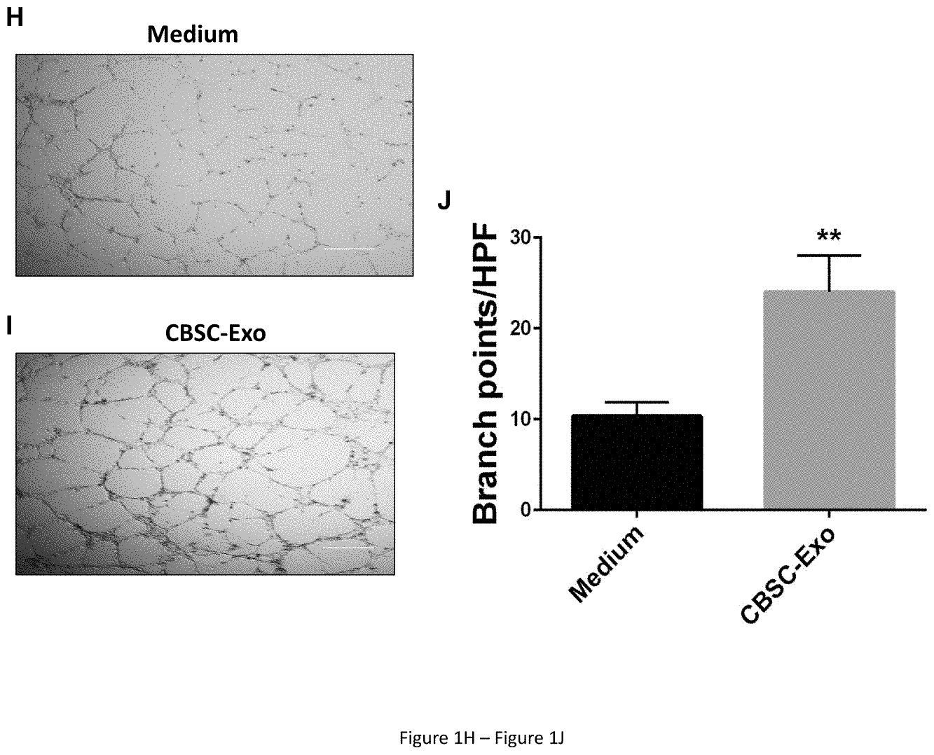

CBSCs were isolated from C57BL/6 mice as described previously (Duran et al., 2013, Circ Res., 113(5):539-52; Mohsin et al., 2015, Circ Res., 117(12):1024-33) and maintained in conditioned medium (base medium+exosomes free FBS). Exosomes were collected from CBSC media by sucrose gradient ultra-centrifugation (Khan et al., 2015, Circ Res., 117(1):52-64). Transmission electron microscopy and Dynamic light scattering (DLS) was used to confirm exosome size (FIG. 1A-FIG. 1C).

The results of the experiments are now described.

### Exosomes Derived From CBSCs Protect Myocytes From Death-Inducing Stimuli and Enhance Tube Formation in HUVECS

Several key hypotheses have surfaced regarding the mechanisms for stem cell mediated improvement of cardiac function after MI (Baraniak et al., 2010, Regen Med., 5(1):121-43). One of the likely mechanisms is protection or salvage of the host myocytes, particularly those myocytes in the MI border zone. Death of myocytes in the area surrounding the MI core leads to infarct expansion and protection of these myocytes should result in improved cardiac function and increased cardiac contractile mass. In-vitro experiments were performed to provide proof of concept that this protection is achieved via exosomes. Neonatal rat ventricular myocytes (NRVMs) were treated with CBSCs and exosomes derived from CBSCs and then exposed to oxidative stress to induce cell death. Treatment with both CBSCs and CBSC-derived exosomes reduced the number of TUNEL positive (apoptotic) NRVMS (FIG. 1D-FIG. 1G).

CBSCs have also been shown to induce angiogenesis in the post MI heart. To determine if CBSC-derived exosomes are involved in this effect we added CBSCs derived exosomes on HUVECS that were plated with matrigel and observed enhanced tube formation, consistent with an angiogenic effect of CBSC exosome contents. (FIG. 1H-FIG. 1J).

### Effect of Exosomes Derived From CBSCs in Mouse MI Models

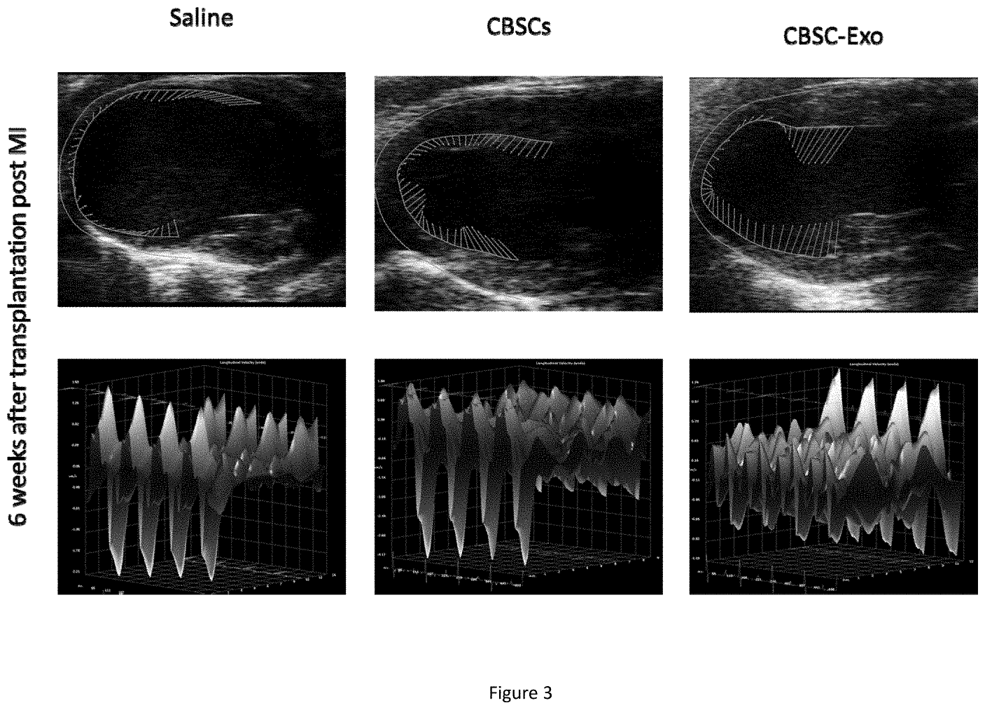

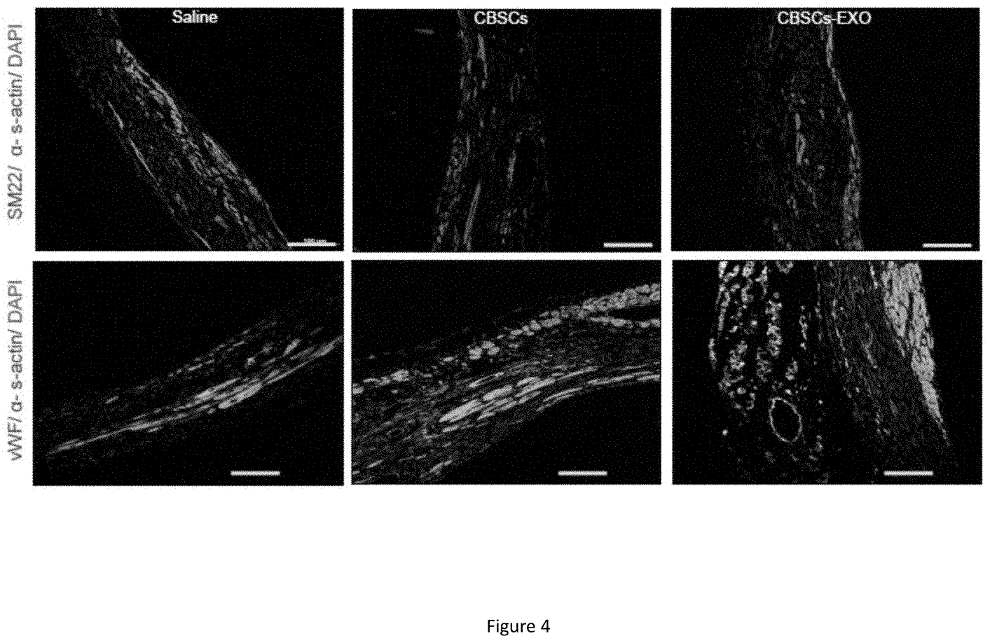

Mouse studies were performed using permanent occlusion MI (Makarewich et al., 2014, Circ Res., 115(6):567-580; Duran et al., 2013, Circ Res., 113(5):539-52). After cardiac injury, exosomes derived from mouse CBSCs were isolated and quantified (Khan et al., 2015, Circ Res., 117(1):52-64). After baseline echocardiography (ECHO) (Duran et al., 2013, Circ Res., 113(5):539-52) an MI was induced (Day 0). Exosomes derived from CBSCs (60-120 μg protein) were directly injected into the MI border zone at the time of MI as described previously (Duran et al., 2013, Circ Res., 113(5):539-52). An EdU containing mini-pump was inserted at the time of MI and removed 7 days later for identification of proliferative cells as described in previous studies (Duran et al., 2013, Circ Res., 113(5):539-52). Animals were sacrificed 1 and 6 weeks after MI. ECHO analysis was performed at these time points in all animals. Studies with the MI injury model suggest that exosomes derived from CBSCs can produce the same beneficial effects as CBSCs in mice after MI (FIG. 2A-FIG. 2B, FIG. 3). Similarly, exosomes derived from CBSCs were shown to have the capacity to form vessels. Mice injected with exosomes have increased vessel density (FIG. 4). Similarly, mice treated with CBSCs or CBSC derived exosomes showed decreased fibrosis versus saline treated animals after MI (FIG. 2C-D). Additionally, CBSCs and CBSCs derived exosomes showed cardioprotection demonstrated by fewer TUNEL stained cells two days post MI (FIG. 2E-FIG. 2F). These results strongly support the idea that exosomes from CBSCs are, at least in part, responsible for the antifibrotic, angiogenic and cardioprotective features of CBSCs.

### CBSCs and CBSCs Derived Exosomes Modulate the Native Immune Response After Cardiac Injury.

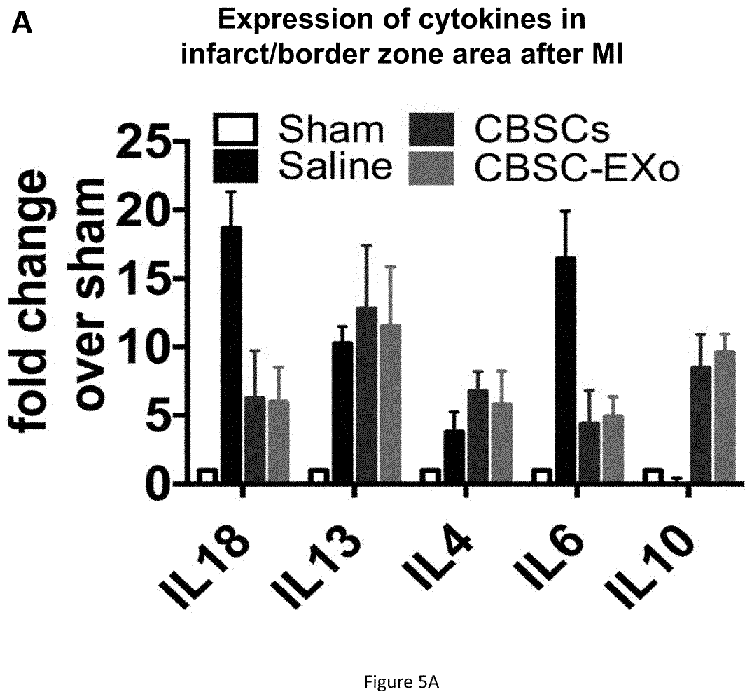

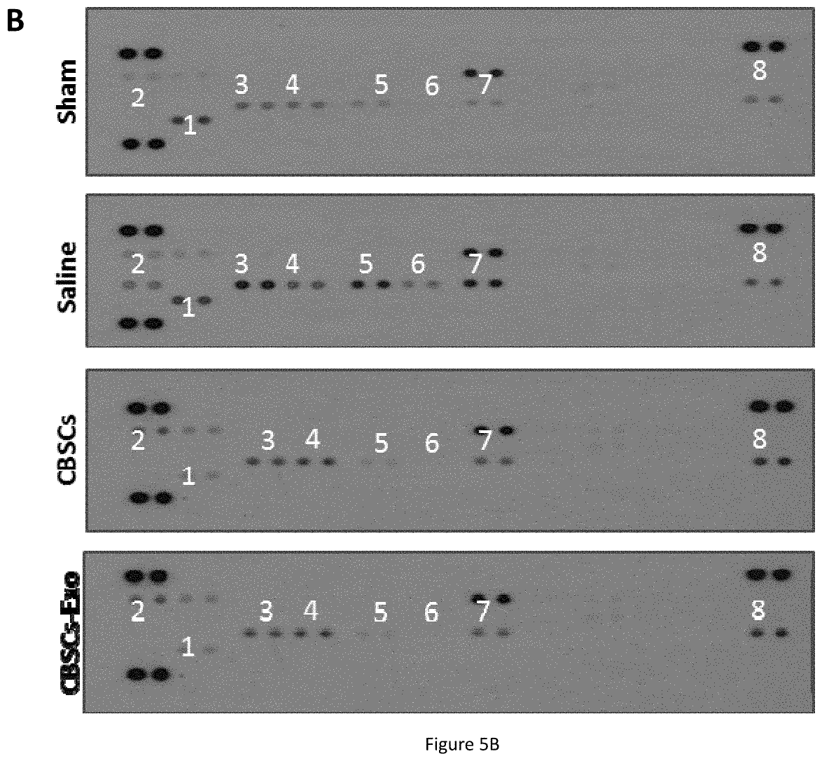

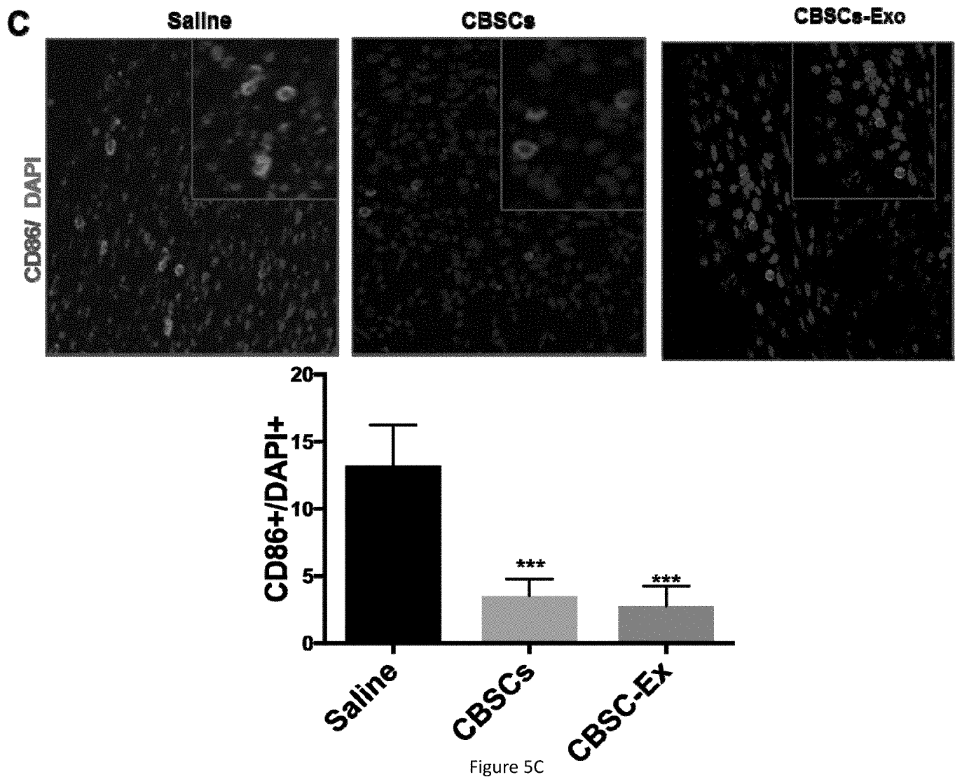

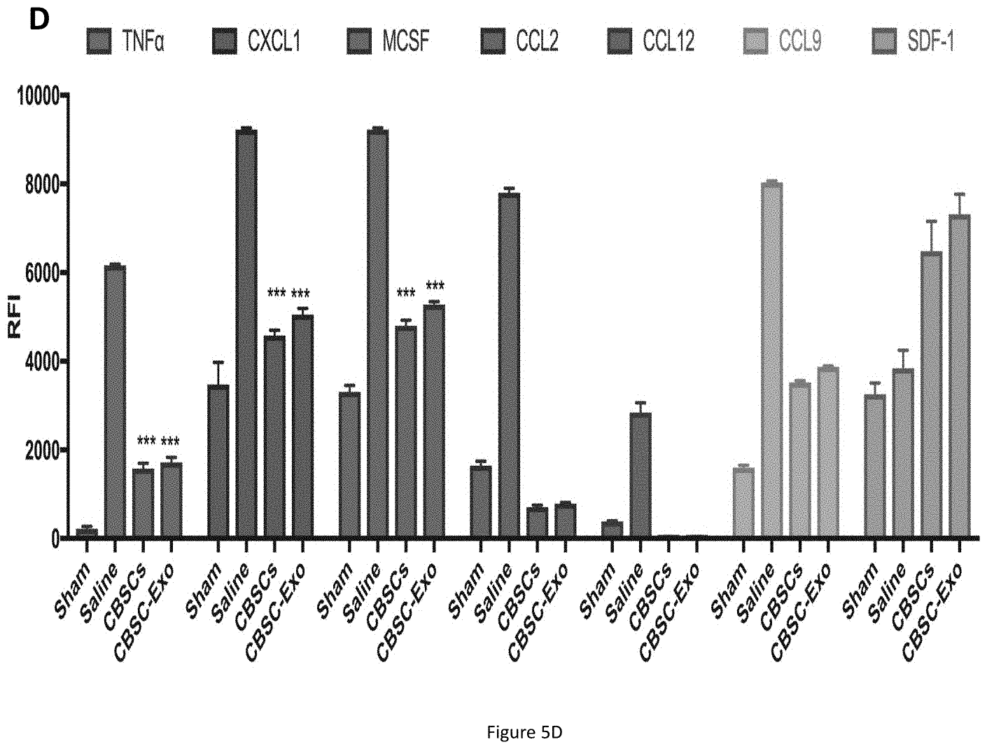

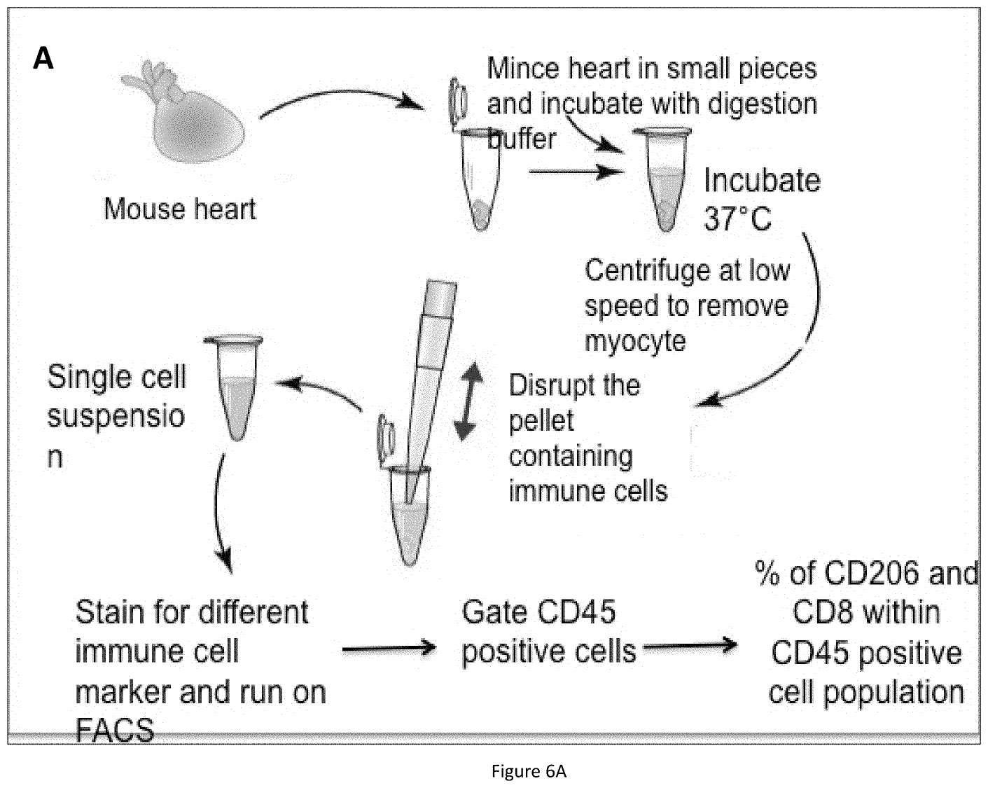

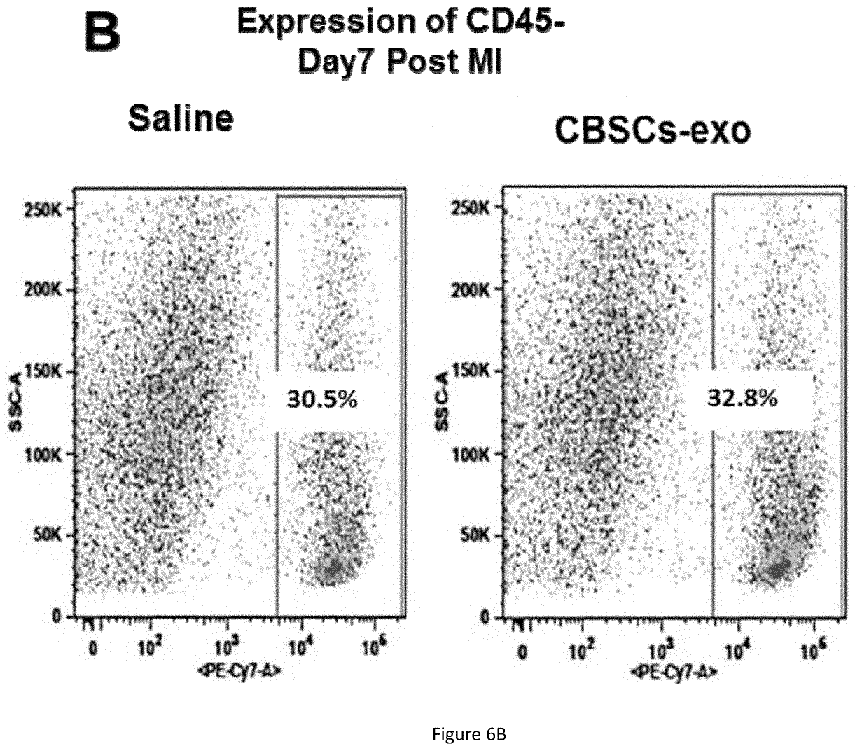

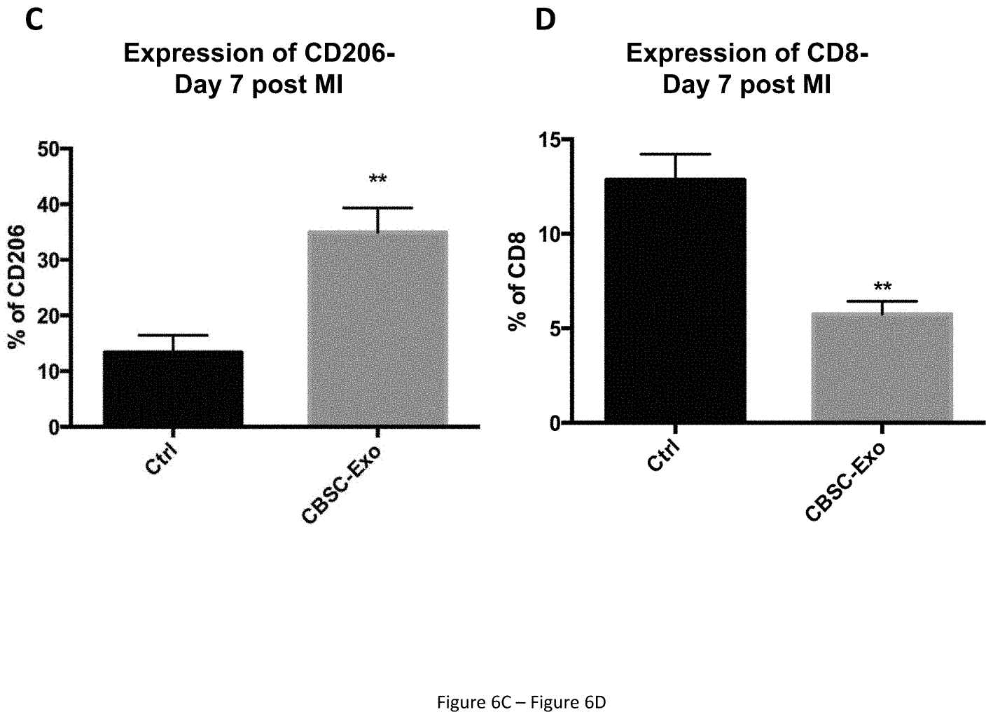

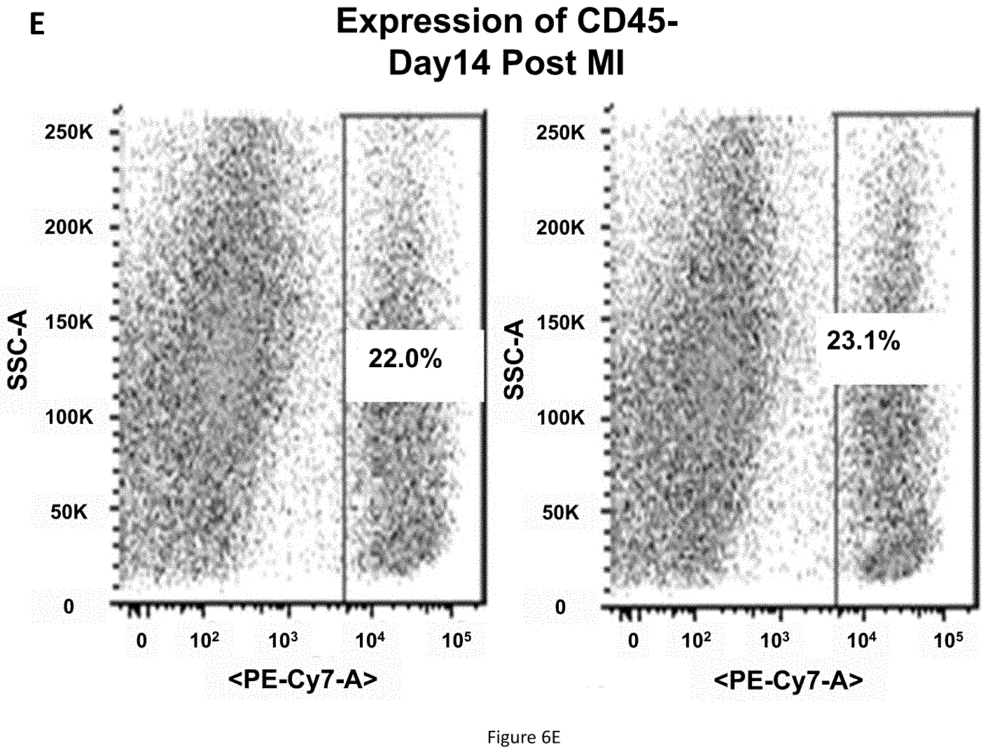

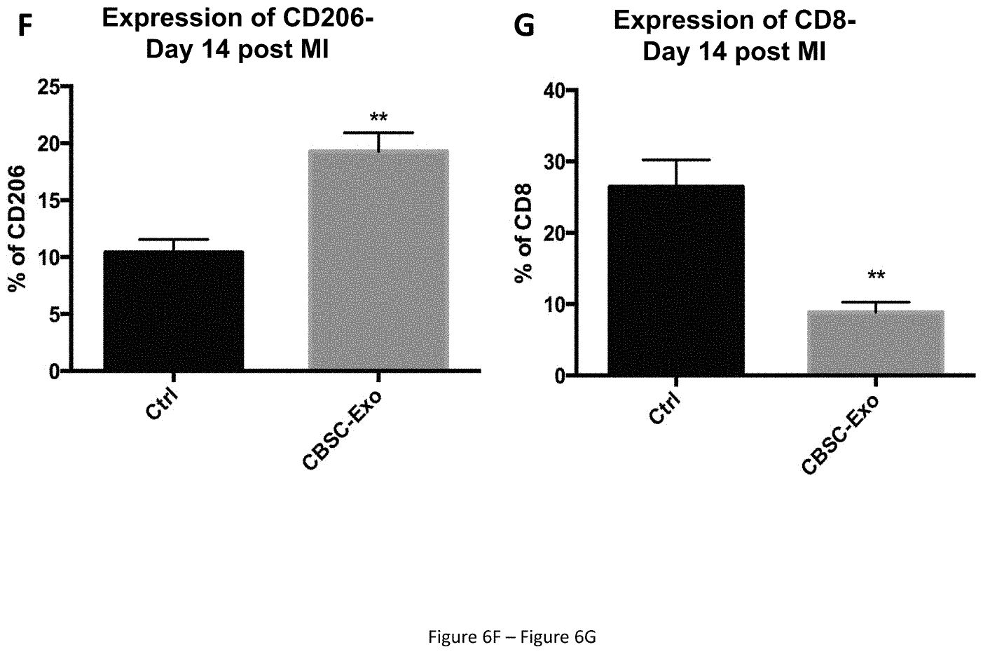

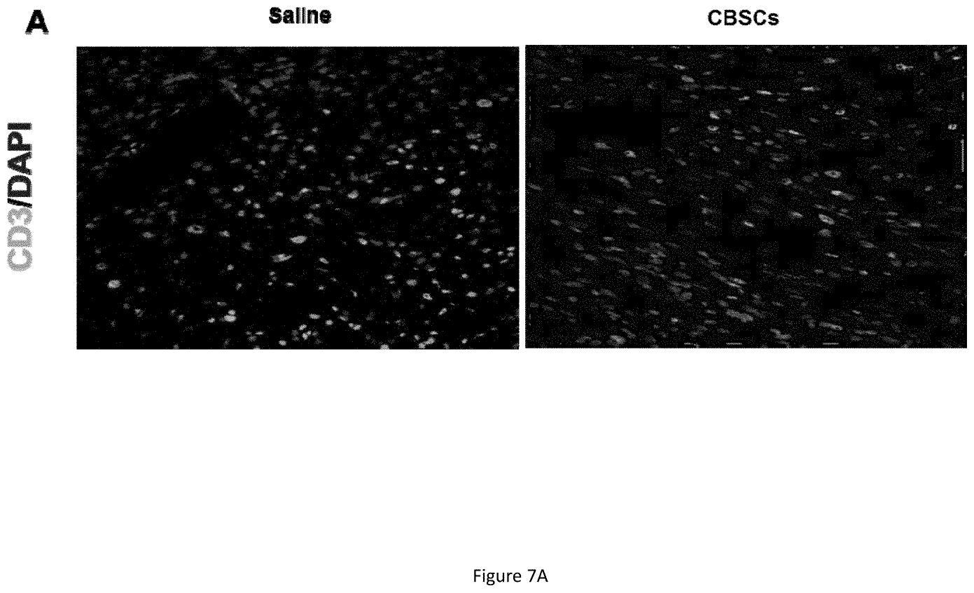

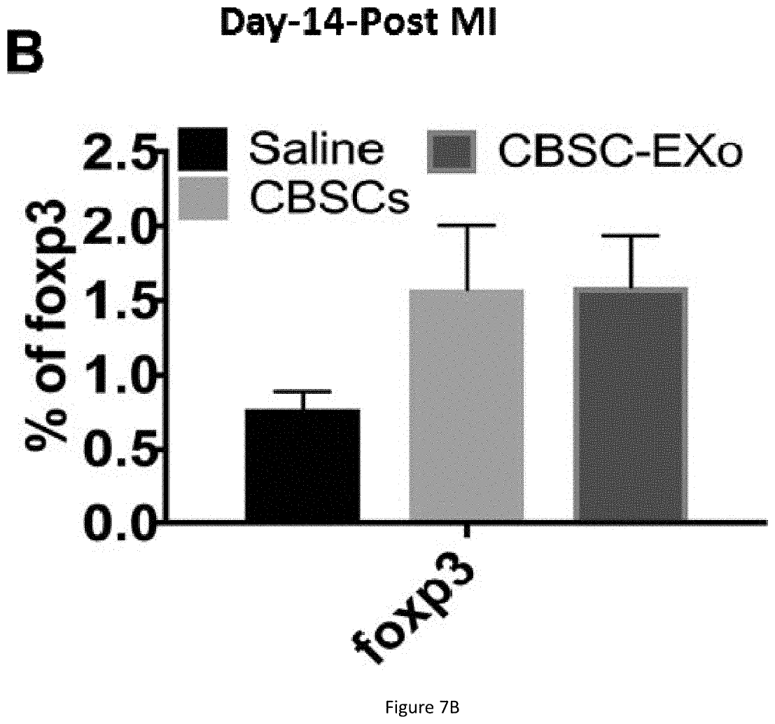

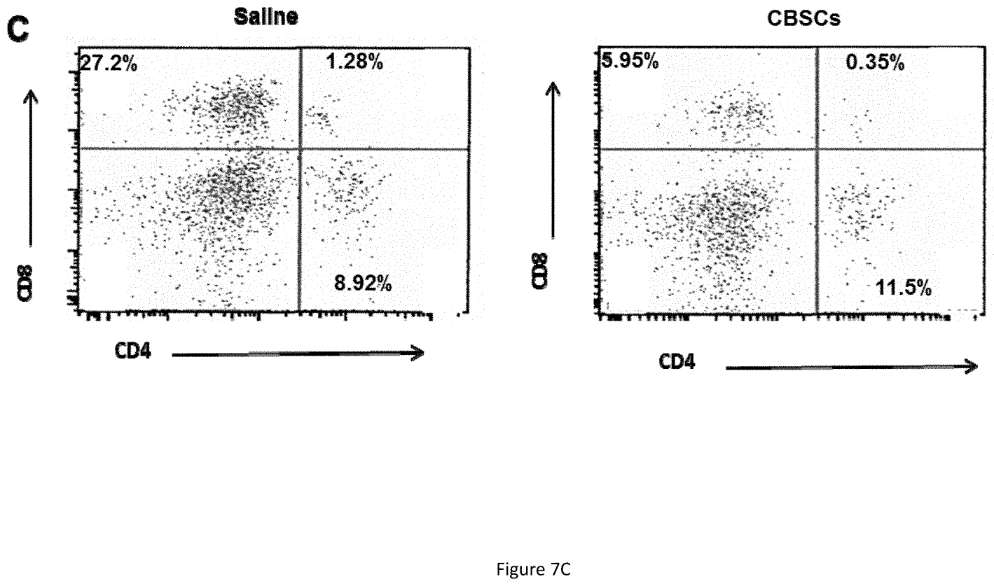

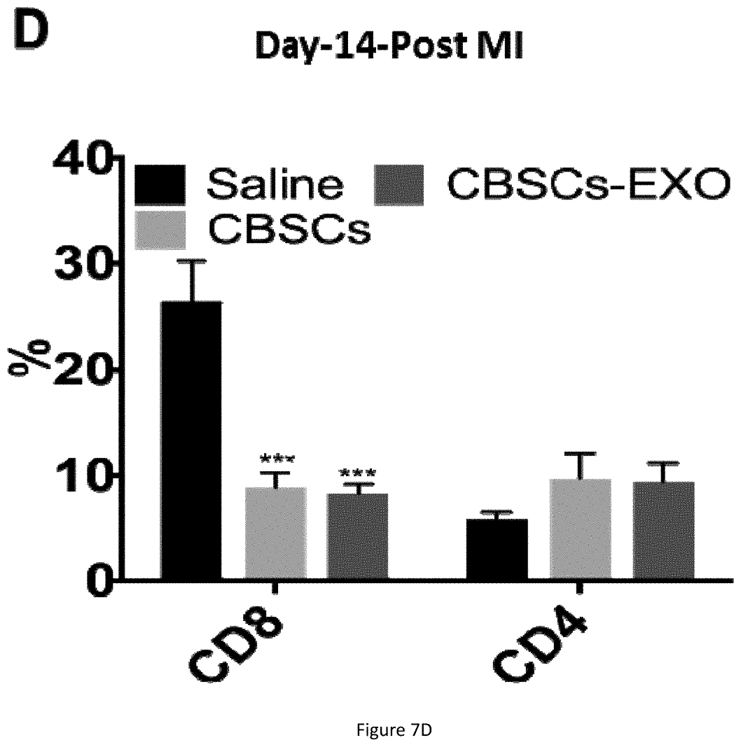

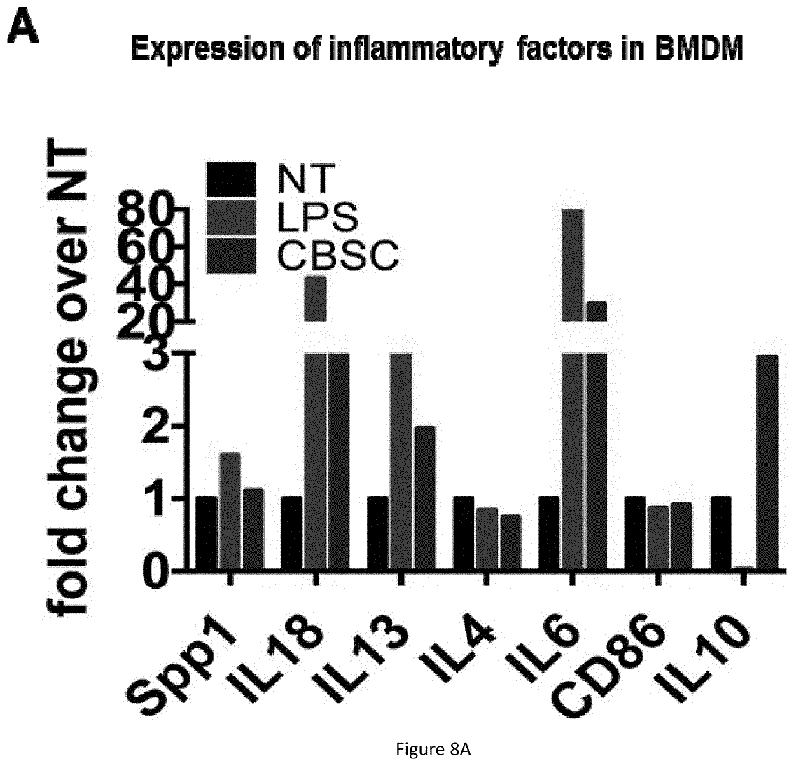

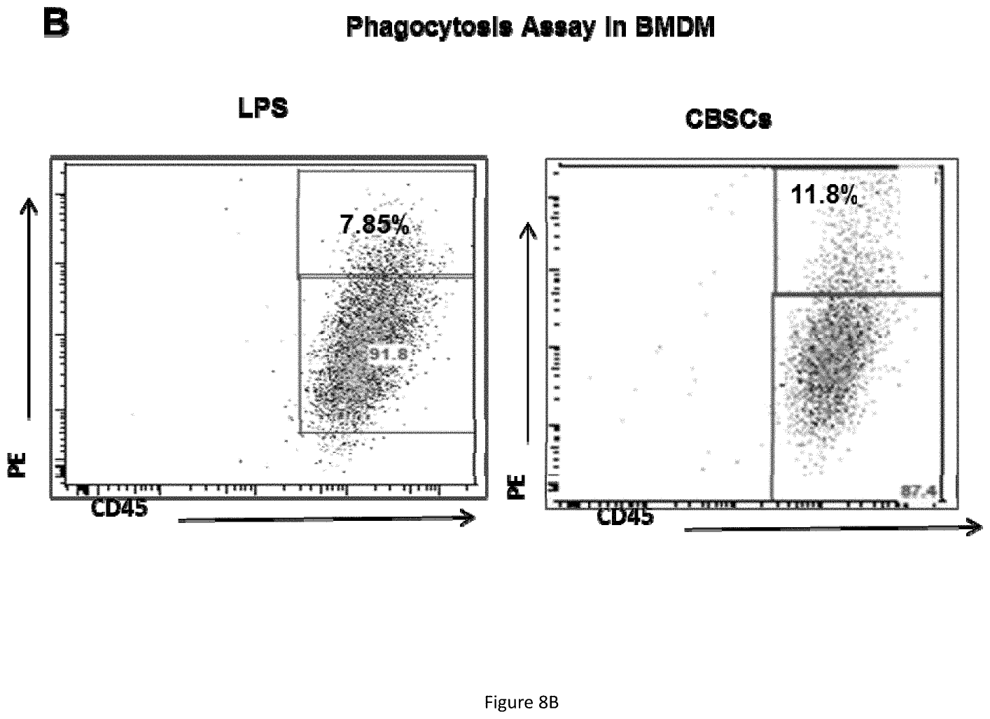

The injured myocardium releases a number of pro-inflammatory factors that attract immune cells that remove dead tissue and then modulate scar formation (Frangogiannis, 2006, Antioxid Redox Signal, 8:1907-1939; Pfeffer and Braunwald, 1990, Circulation 81:1161-1172). The idea that an effect of transplanted stem cells on the cardiac inflammatory/immune response is involved in their reparative properties is an important research area. The data presented herein suggests that CBSCs secretome consists of cardioprotective factors with the ability to modulate cardiac immune response enhancing repair after injury. Histological analysis showed 3.7 fold decreased expression of CD86 (marker for pro-inflammatory macrophage) after CBSCs treatment (FIG. 5C) compared to saline treated animals 7 days post MI. Moreover, serum analysis of CBSCs and CBSCs-exosome treated animals showed significantly less expression of pro-inflammatory cytokines including TNF-α, CXCL10, CXCL1 CCL2, CCL12 CCL9 compared to saline controls as measured by cytokine profiler array (R&D systems) (FIG. 5B and FIG. 5D). In parallel, expression of SDF-1 and M-CSF was elevated in CBSCs animals. These findings were further validated by mRNA expression analysis that showed significant reduction of pro and increased expression of anti-inflammatory factors in border zone of animals treated with CBSCs and CBSCs derived exosomes (FIG. 5A). Additionally, expression of pan-hematopoietic marker CD45 remained unchanged between CBSCs derived exosomes and saline administered animals 7 days (FIG. 6B) and 14 days (FIG. 6E) post MI measured by FACS analysis suggesting that CBSC derived exosomes may not change the overall cell number but rather shift the balance of immune cell subsets towards anti-inflammatory state (FIG. 6C-FIG. 6D and FIG. 6F-FIG. 6G). Collectively, CBSC exosomes enhance cardiac function by promoting cardiomyocyte survival and modulating the cardiac immune response in the heart post-MI. The strategy for isolation of cardiac immune cells is delineated in FIG. 6A. Expression of CD3+ cells was decreased after CBSCs transplantation in the heart 14 days post MI (FIG. 7A). Similarly, expression of CD4+ positive cells was increased along with decrease in the number of CD8+ T cell subset as measured by FACS analysis in CBSCs and CBSCs-Exo transplanted animals versus saline treated animals (FIG. 7C-D). Interestingly, there is an increase in foxp3+ cells in CBSCs and CBSCs-Exo hearts (FIG. 7B). To test the capacity of immune modulation in vitro, isolated macrophages from the bone marrow (BMDMΦs) were co-cultured in a trans-well system with CBSCs in the presence of LPS for 4 hours while untreated BMDMΦs were used as controls. CBSCs induced significant reduction of pro-inflammatory factors in BMDMΦs compared to LPS treatment parallel with increased expression of anti-inflammatory cytokine IL-10 (FIG. 8A) indicating the ability of CBSCs cardioprotective factors to promote anti-inflammatory phenotype in BMDMΦs. Concurrently, increased uptake of PE conjugated phagocytosis beads was observed in BMDMΦs treated with CBSCs medium compared to LPS treatment (FIG. 8B). Collectively; these data suggest that CBSCs and CBSCs derived exosomes modulate cardiac immune cells response in the heart after myocardial injury.

### Exosomes Carry Signature Cardioprotective Cargo of Parent CBSCs

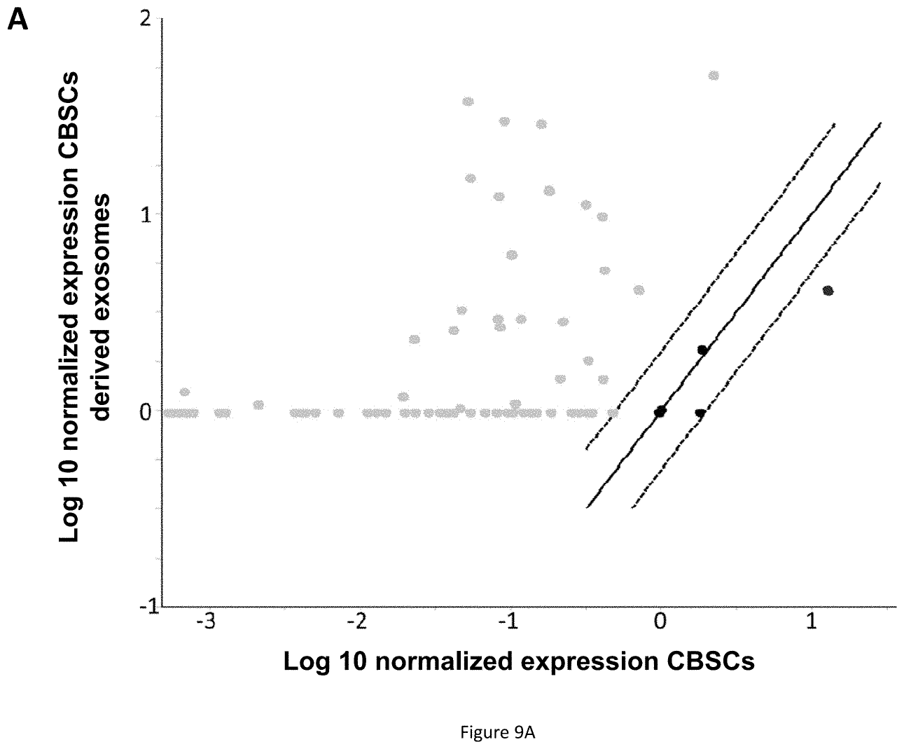

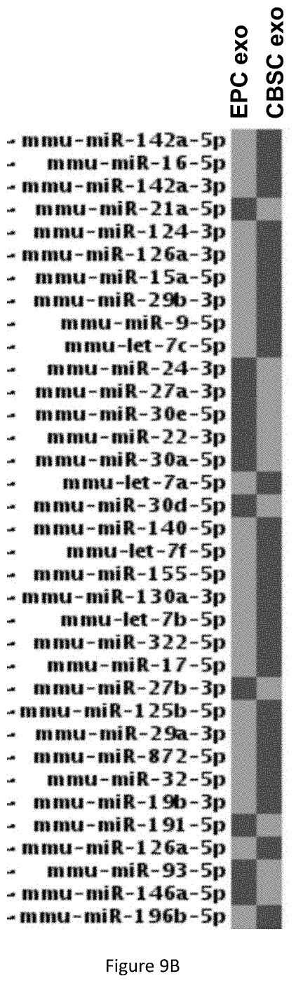

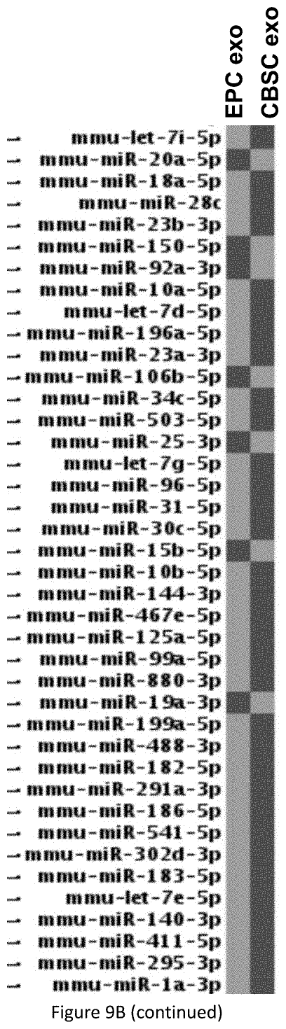

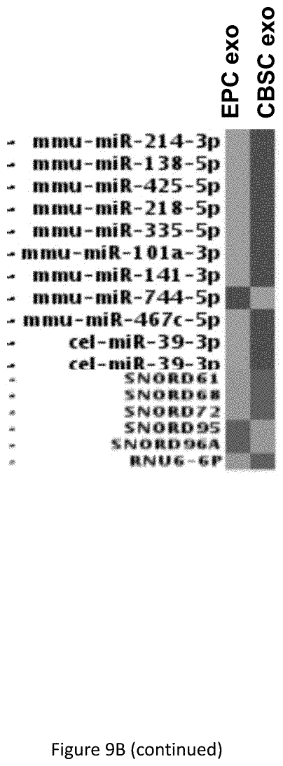

It is extremely important to determine if exosomes derived from CBSCs carry cardioprotective factors responsible for beneficial effects observed in previous studies. CBSCs are novel stem cells packed with cardioreparative paracrine factors (Mohsin et al., 2015, Circ Res., 117(12):1024-33). Recent studies suggest that injected cells disappear a few days after injection (Gallina et al., 2015, Stem Cells Int., 2015:765846) into the damaged heart. Therefore, the beneficial effects may be due to the presence of exosomes derived from these cells. The present data, obtained by comparing CBSCs (parent cell) with exosomes derived from CBSCs, showed packaging of miRs in exosomes (FIG. 9A, FIG. 9B). Exosomes derived from CBSCs were compared to other stem cell type Endothelial Progenitor Cells (EPC)-derived exosomes, and they contain different types and amounts of miRs versus CBSC-derived exosomes (FIG. 9B). These results show the CBSC-derived exosomes carry a unique set of molecules that induce specific effects in the post MI heart.

### Effect of Exosomes Derived From CBSCs in a Pig IR MI Model

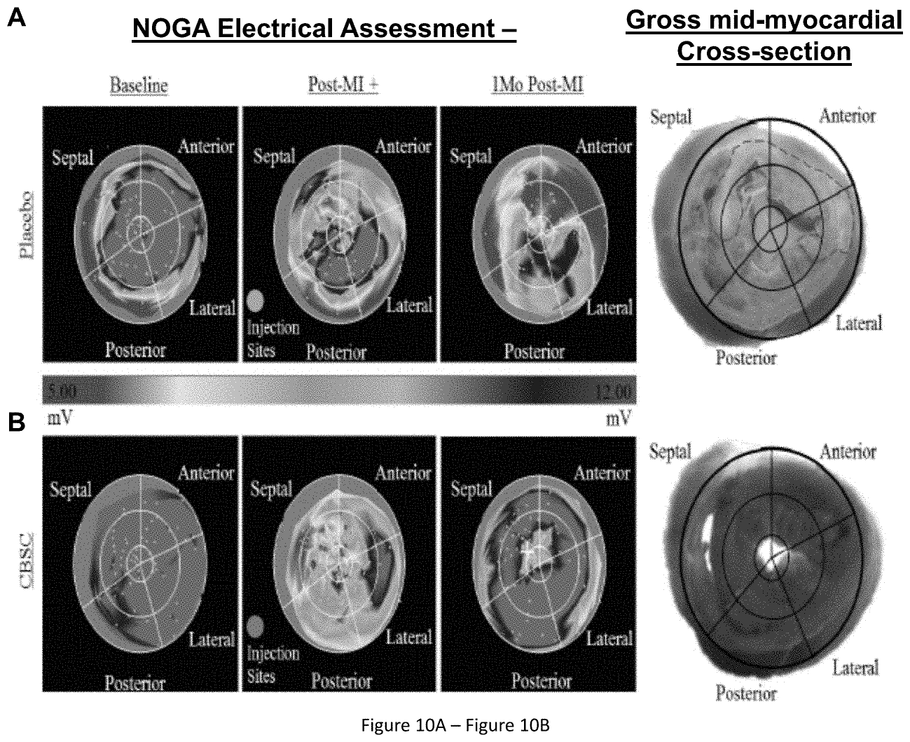

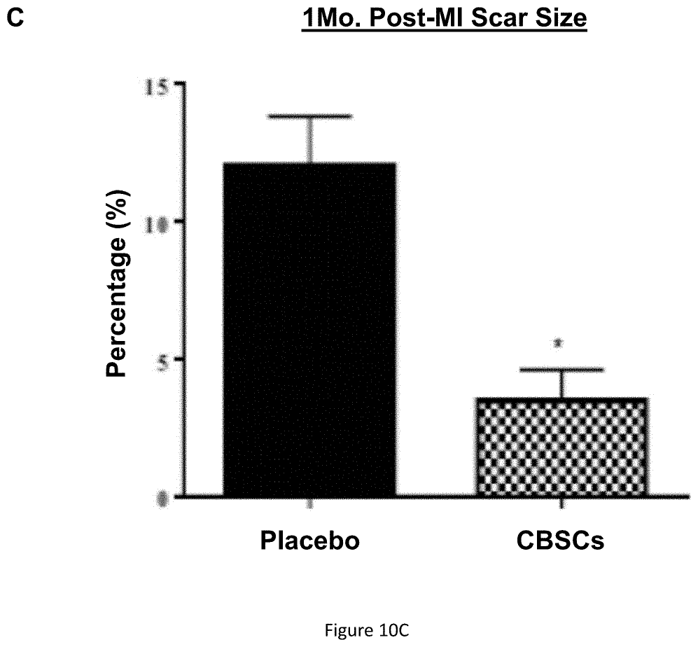

To develop therapies that improve cardiac function in patients, rather than in rodents, CBSCs were injected in a blinded fashion in a large animal model in a clinically relevant setting. The techniques for induction of MI have been established in a large animal core laboratory (Khan et al., 2015, Circ Res., 117(1):52-64; Baraniak et al., 2010, Regen Med., 5(1):121-43). Briefly, MI was induced by percutaneous insertion of a balloon catheter that is used to occlude the left anterior descending artery, just after the first diagonal branch, for 90-120 minutes in miniswine. Balloon occlusion was confirmed by angiography. After the occlusion period the balloon was deflated and the animal was allowed to recover. Sham animals had the same procedure except that the balloon was not inflated. These techniques have been used in other recent studies. Cardiac structure and function was assessed with ECHO, including regional strain analysis, and invasive hemodynamic (pressure and volume) measurements made before and after MI. The infarction size was determined by NOGA mapping (FIG. 10A, FIG. 10B) of the endocardial surface of the left ventricle after MI induction. The post MI NOGA map was used to guide injections of CBSC into the MI border zone. Ten injections were performed around the border of the infarct as described (Taghavi et al., 2012, Am J Transl Res., 4(2):240-6). Animals were evaluated for 1 month after MI. Animals injected with CBSCs showed decrease in scar size (FIG. 10C).

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A composition for treating a cardiovascular disease or disorder in a subject, comprising an isolated cortical bone stem cell (CBSC)-derived exosome.

2. The composition of claim 1, further comprising at least one RNA molecule.

3. The composition of claim 2, wherein said RNA molecule is at least one selected from the group consisting of: miR-142, miR-16, miR-21, miR-124, miR-126, miR-15, miR-29, miR-9, let-7, miR-24, miR-27, miR-30, miR-22, miR-140, miR-155, miR-130, miR-322, miR-17, miR-125, miR-29, miR-872, miR-32, miR-19, miR-191, miR-126, miR-93, miR-146, miR-196, miR-30, miR-18, miR-28, miR-23, miR-150, miR-92, miR-10, miR-106, miR-34, miR-503, miR-25, miR-96, miR-31, miR-15, miR-10, miR-144, miR-467, miR-99, miR-880, miR-199, miR-488, miR-182, miR-291, miR-186, miR-541, miR-302, miR-183, miR-411, miR-295, miR-1, miR-214, miR-138, miR-425, miR-218, miR-335, miR-101, miR-141, miR-744, miR-39, miR-142a-5p, miR-16-5p, miR-142a-3p, miR-21a-5p, miR-124-3p, miR-126a-3p, miR-15a-5p, miR-29b-3p, miR-9-5p, let-7c-5p, miR-24-3p, miR-27a-3p, miR-30e-5p, miR-22-3p, miR-30a-5p, let-7a-5p, miR-30d-5p, miR-140-5p, let-7f-5p, miR-155-5p, miR-130a-3p, let-7b-5p, miR-322-5p, miR-17-5p, miR-27b-3p, miR-125b-5p, miR-29a-3p, miR-872-5p, miR-32-5p, miR-19b-3p, miR-191-5p, miR-126a-5p, miR-93-5p, miR-146a-5p, miR-196b-5p, let-7i-5p, miR-20a-5p, miR-18a-5p, miR-28c, miR-23b-3p, miR-150-5p, miR-92a-3p, miR-10a-5p, let-7d-5p, miR-196a-5p, miR-23a-3p, miR-106b-5p, miR-34c-5p, miR-503-5p, miR-25-3p, miR-7g-5p, miR-96-5p, miR-31-5p, miR-30c-5p, miR-15b-5p, miR-10b-5p, miR-144-3p, miR-467e-5p, miR-125a-5p, miR-99a-5p, miR-880-3p, miR-19a-3p, miR-199a-5p, miR-488-3p, miR-182-5p, miR-291a-3p, miR-186-5p, miR-541-5p, miR-302d-3p, miR-183-5p, let-7e-5p, miR-140-3p, miR-411-5p, miR-295-3p, miR-1a-3p, miR-214-3p, miR-138-5p, miR-425-5p, miR-218-5p, miR-335-5p, miR-101a-3p, miR-141-3p, miR-744-5p, miR-467c-5p, miR-39-3p, SNORD61, SNORD68, SNORD72, SNORD95, SNORD96A, RNU6-6P, a variant, a derivative, and a combination thereof.

4. The composition of claim 1, further comprising a pharmaceutically acceptable excipient, carrier, or diluent.

5. A composition for treating a cardiovascular disease or disorder in a subject, comprising at least one RNA molecule.

6. The composition of claim 5, wherein said RNA molecule is at least one selected from the group consisting of: miR-142, miR-16, miR-21, miR-124, miR-126, miR-15, miR-29, miR-9, let-7, miR-24, miR-27, miR-30, miR-22, miR-140, miR-155, miR-130, miR-322, miR-17, miR-125, miR-29, miR-872, miR-32, miR-19, miR-191, miR-126, miR-93, miR-146, miR-196, miR-30, miR-18, miR-28, miR-23, miR-150, miR-92, miR-10, miR-106, miR-34, miR-503, miR-25, miR-96, miR-31, miR-15, miR-10, miR-144, miR-467, miR-99, miR-880, miR-199, miR-488, miR-182, miR-291, miR-186, miR-541, miR-302, miR-183, miR-411, miR-295, miR-1, miR-214, miR-138, miR-425, miR-218, miR-335, miR-101, miR-141, miR-744, miR-39, miR-142a-5p, miR-16-5p, miR-142a-3p, miR-21a-5p, miR-124-3p, miR-126a-3p, miR-15a-5p, miR-29b-3p, miR-9-5p, let-7c-5p, miR-24-3p, miR-27a-3p, miR-30e-5p, miR-22-3p, miR-30a-5p, let-7a-5p, miR-30d-5p, miR-140-5p, let-7f-5p, miR-155-5p, miR-130a-3p, let-7b-5p, miR-322-5p, miR-17-5p, miR-27b-3p, miR-125b-5p, miR-29a-3p, miR-872-5p, miR-32-5p, miR-19b-3p, miR-191-5p, miR-126a-5p, miR-93-5p, miR-146a-5p, miR-196b-5p, let-7i-5p, miR-20a-5p, miR-18a-5p, miR-28c, miR-23b-3p, miR-150-5p, miR-92a-3p, miR-10a-5p, let-7d-5p, miR-196a-5p, miR-23a-3p, miR-106b-5p, miR-34c-5p, miR-503-5p, miR-25-3p, miR-7g-5p, miR-96-5p, miR-31-5p, miR-30c-5p, miR-15b-5p, miR-10b-5p, miR-144-3p, miR-467e-5p, miR-125a-5p, miR-99a-5p, miR-880-3p, miR-19a-3p, miR-199a-5p, miR-488-3p, miR-182-5p, miR-291a-3p, miR-186-5p, miR-541-5p, miR-302d-3p, miR-183-5p, let-7e-5p, miR-140-3p, miR-411-5p, miR-295-3p, miR-1a-3p, miR-214-3p, miR-138-5p, miR-425-5p, miR-218-5p, miR-335-5p, miR-101a-3p, miR-141-3p, miR-744-5p, miR-467c-5p, miR-39-3p, SNORD61, SNORD68, SNORD72, SNORD95, SNORD96A, RNU6-6P, a variant, a derivative, and a combination thereof.

7. The composition of claim 5, further comprising a pharmaceutically acceptable excipient, carrier, or diluent.

8. A method of treating at least one cardiovascular disease or disorder in a subject, comprising administering to said subject a therapeutically effective amount of a composition comprising at least one selected from the group consisting of: a CBSC-derived exosome and an RNA molecule.

9. The method of claim 8, wherein said RNA molecule is at least one selected from the group consisting of: miR-142, miR-16, miR-21, miR-124, miR-126, miR-15, miR-29, miR-9, let-7, miR-24, miR-27, miR-30, miR-22, miR-140, miR-155, miR-130, miR-322, miR-17, miR-125, miR-29, miR-872, miR-32, miR-19, miR-191, miR-126, miR-93, miR-146, miR-196, miR-30, miR-18, miR-28, miR-23, miR-150, miR-92, miR-10, miR-106, miR-34, miR-503, miR-25, miR-96, miR-31, miR-15, miR-10, miR-144, miR-467, miR-99, miR-880, miR-199, miR-488, miR-182, miR-291, miR-186, miR-541, miR-302, miR-183, miR-411, miR-295, miR-1, miR-214, miR-138, miR-425, miR-218, miR-335, miR-101, miR-141, miR-744, miR-39, miR-142a-5p, miR-16-5p, miR-142a-3p, miR-21a-5p, miR-124-3p, miR-126a-3p, miR-15a-5p, miR-29b-3p, miR-9-5p, let-7c-5p, miR-24-3p, miR-27a-3p, miR-30e-5p, miR-22-3p, miR-30a-5p, let-7a-5p, miR-30d-5p, miR-140-5p, let-7f-5p, miR-155-5p, miR-130a-3p, let-7b-5p, miR-322-5p, miR-17-5p, miR-27b-3p, miR-125b-5p, miR-29a-3p, miR-872-5p, miR-32-5p, miR-19b-3p, miR-191-5p, miR-126a-5p, miR-93-5p, miR-146a-5p, miR-196b-5p, let-7i-5p, miR-20a-5p, miR-18a-5p, miR-28c, miR-23b-3p, miR-150-5p, miR-92a-3p, miR-10a-5p, let-7d-5p, miR-196a-5p, miR-23a-3p, miR-106b-5p, miR-34c-5p, miR-503-5p, miR-25-3p, miR-7g-5p, miR-96-5p, miR-31-5p, miR-30c-5p, miR-15b-5p, miR-10b-5p, miR-144-3p, miR-467e-5p, miR-125a-5p, miR-99a-5p, miR-880-3p, miR-19a-3p, miR-199a-5p, miR-488-3p, miR-182-5p, miR-291a-3p, miR-186-5p, miR-541-5p, miR-302d-3p, miR-183-5p, let-7e-5p, miR-140-3p, miR-411-5p, miR-295-3p, miR-1a-3p, miR-214-3p, miR-138-5p, miR-425-5p, miR-218-5p, miR-335-5p, miR-101a-3p, miR-141-3p, miR-744-5p, miR-467c-5p, miR-39-3p, SNORD61, SNORD68, SNORD72, SNORD95, SNORD96A, RNU6-6P, a variant, a derivative, and a combination thereof.

10. The method of claim 8, wherein said cardiovascular disease is myocardial injury.

11. The method of claim 10, wherein said myocardial injury is at least one selected from the group consisting of: arterial disease, atheroma, atherosclerosis, arteriosclerosis, coronary artery disease, arrhythmia, angina pectoris, congestive heart disease, ischemic cardiomyopathy, myocardial infarction, stroke, transient ischemic attack, aortic aneurysm, cardiopericarditis, infection, inflammation, valvular insufficiency, vascular clotting defects, and a combination thereof.

12. The method of claim 8, wherein said composition is administered to said subject by at least one selected from the group consisting of: direct injection, venous infusion, and arterial infusion.

13. The method of claim 8, wherein said composition further comprises a pharmaceutically acceptable excipient, carrier, or diluent.
